(19) United States
(12) Patent Application Publication
(10) Pub. No.: US 2003/0113327 A1
(43) Pub. Date: Jun. 19, 2003

(54) Compositions and methods for adipose abundant protein

(76) Inventor: Sean H. Adams, Randolph Township, NJ (US)

Correspondence Address: SONNENSCHEIN NATH & ROSENTHAL, P.O. BOX 061080, WACKER DRIVE STATION, CHICAGO, IL, 60606-1080, US

(21) Appl. No.: 10/155,895

(22) Filed: May 24, 2002

Related U.S. Application Data
(60) Provisional application No. 60/293,775, filed on May 25, 2001.

(51) Int. Cl. A61K 39/395; C07H 21/04; C12N 9/20; C12P 21/02; C12N 5/06; C07K 14/435; C07K 16/40

(52) U.S. Cl. 424/146.1; 435/69.1; 435/198; 435/320.1; 435/325; 536/23.2; 530/388.26

(57) ABSTRACT

An isolated polypeptide comprising an amino acid sequence having at least 80% sequence identity to the sequence SEQ ID NOS:4 or 6, polynucleotides encoding these peptides, and antibodies to the polypeptides are useful in treating metabolic disorders or disorders associated with changes in adipose tissue physiological function or mass.

### BACKGROUND

Metabolic diseases represent a serious public health concern worldwide. Obese and overweight individuals account for up to half of the population of the United States, and the incidence of obesity has risen at an alarming rate over the last decade. Compared to lean individuals, overweight persons are particularly susceptible to an array of disorders, including heart disease, high blood pressure, Type II diabetes/insulin resistance, stroke, and others (Must et al., 1999). The etiology of excess weight gain is complex and incompletely understood, but in general, approximately equal contribution of genetics and environmental/social factors occurs with respect to weight gain or loss (Bray, 1997; Hill and Peters, 1998). In persons developing overweight or obese conditions, these factors underlie an imbalance between appetite/caloric intake and energy expenditure. Thus, novel strategies that improve energy balance through modulation of appetite and/or metabolic rate are useful to correct relevant diseases including obesity-related disorders such as insulin resistance/diabetes, circulatory system anomalies, etc. Strategies to thwart excessive adipose tissue growth and accretion may also help in the treatment of obesity, or in the treatment of other diseases associated with the presence of inappropriately high amounts of this tissue globally or locally (i.e., lipomas, phaeochromocytomas, hibernomas). Innovative clinical treatments, including improving the circulating or tissue levels of triglycerides, cholesterol, glucose, insulin, leptin or other metabolically-relevant molecules, that help normalize one or more of the altered factors concomitant with metabolic derangement would also have tremendous value.

Increased risks of mortality and morbidity associated with perturbations of metabolism are not confined to the obese, overweight, or diabetic states, however. Cachexia, the loss of appetite, leading to fewer calories taken in compared to caloric requirements, is a feature of numerous disease states, including certain cancers, some viral infections (i.e., acquired immunodeficiency syndrome, AIDS), or bacterial infections (i.e., during some stages of sepsis). The clinical prognosis is poor for patients who drift into negative energy balance (Tisdale, 1997), and thus there is a need for new treatments that counteract cachexia. Furthermore, conditions in which energy expenditure is abnormally elevated can benefit from novel modalities that modulate metabolism. In severe burns, for instance, the metabolic rate can rise almost two-fold, making administration of appropriate nutrition a tremendous challenge (Goldstein and Elwyn, 1989; Kinney et al., 1970). Finally, poor outcomes from diseases characterized by excessive adipose tissue loss (i.e., lipoatrophic disorders) are common.

Treatment of metabolic disease may be in part effected through the innovative use of certain molecules as drugs or as targets of pharmaceutical intervention. However, there is also a pressing need to discover molecules that may be used in creative diagnostic and/or predictive strategies associated with metabolic disease. For instance, alterations in the expression of certain genes and proteins may underlie or mark the progression of metabolic diseases such as obesity. Thus, analysis of the expression of certain genes and proteins in afflicted patients compared to a normal population will assist in unraveling the etiology of their disease, thereby helping in the design of effective therapeutic strategies. Normal patients may be screened for expression in cases in which expression is altered prior to the onset of disease, thus allowing for pre-emptive treatments that can limit metabolic or other disease progression. Furthermore, changes in the gene or protein sequences in certain populations may be associated with disease, hence illustrating the need to discover genes/proteins relevant to metabolic disorders and whose sequences lead to biological changes that predispose to metabolic disease, or are in fact predictive of the progression of disease. Finally, knowledge of such unique genes/proteins will enable tracking of the efficacy of therapeutic modalities designed to treat metabolic diseases. The same case may be made for the use of expression and sequence analysis to monitor or predict the outcome of other diseases associated with excessive fat accretion, or those associated with abnormal fat loss. Discovery of fat-specific genes and proteins are especially attractive in this regard.

The current invention outlines the discovery of the novel protein adipose abundant protein (AAP), and use of the protein and AAP-encoding nucleic acids in treating or preventing metabolic diseases in humans. Novel evidence for an important function for AAP in modifying adipose tissue function is presented. The white and brown adipose tissue of mammals are known to play a central role in energy storage and metabolic signaling, thus impacting appetite, metabolic rate, and energy balance. Based on its adipose-abundance and its clear induction during the white adipocyte differentiation process, AAP appears to have important utility as a drug and drug target to treat metabolic disease and related disorders, including those characterized by excessive adipose accretion or abnormally low adipose stores. Furthermore, AAP sequence, mRNA, AAP protein abundance in patient tissues may be used to diagnose, monitor, and assess treatment efficacy of metabolic disorders associated with changes in AAP protein or mRNA levels.

### SUMMARY

Although methods and materials similar or equivalent to those described herein can be used in the practice or testing of the present invention, suitable methods and materials are described below. In the case of conflict, the present specification, including definitions, will control. In addition, the materials, methods, and examples are illustrative only and not intended to be limiting.

### DETAILED DESCRIPTION

A gene encoding an adipose-abundant protein, termed AAP (adipose abundant protein) has been discovered. This gene is strongly and specifically expressed in brown adipose and white adipose tissue, and in the development of pre-adipocytes to adipocytes. The important role of AAP in fat tissues is evidenced by this strong and specific expression.

Altering the expression of AAP in a mammal, such as a human, through gene therapy may be effective to combat metabolic diseases, such as obesity or cachexia, or to increase or decrease body weight. The term “metabolic disease” means a condition in which a disruption of normal energy homeostasis is a causative or exacerbating factor underlying disease. For example, metabolic disease includes obesity, wasting disorders such cachexia (associated with, for example, HIV infection, sepsis and trauma, and cancer), and diabetes (including Type II diabetes and insulin resistance).

Because of the important role of AAP in fat tissues and metabolism, compounds that have the property of increasing or decreasing AAP activity are useful. This increase in activity may come about in a variety of ways, for example: (1) by increasing or decreasing the copies of the gene in the cell (amplifiers and deamplifiers); (2) by increasing or decreasing transcription of the AAP gene (transcription up-regulators and down-regulators); (3) by increasing or decreasing the translation of AAP mRNA into protein (translation up-regulators and down-regulators); or (4) by increasing or decreasing the activity of AAP itself (agonists and antagonists).

Contacting cells or organisms with the compound may identify compounds that are amplifiers and deamplifiers, and then measuring the amount of DNA present that encodes AAP (Ausubel et al., 1987). Contacting cells or organisms with the compound may identify compounds that are transcription up-regulators and down-regulators, and then measuring the amount of mRNA produced that encodes AAP (Ausubel et al., 1987). Compounds that are translation up-regulators and down-regulators may be identified by contacting cells or organisms with the compound, and then measuring the amount of AAP polypeptide produced (Ausubel et al., 1987).

Compounds that are amplifiers, transcription up-regulators, translation up-regulators or agonists, are effective to combat metabolic diseases that can be ameliorated by decreasing AAP activity, such as obesity or to decrease weight gain. Conversely, compounds that are deamplifiers, transcription down-regulators, translation down-regulators or antagonists, are effective to combat metabolic diseases that can be ameliorated by increasing AAP activity, such as cachexia, or to increase weight gain. Gene therapy is another way to up-regulate or down-regulate transcription and/or translation.

Both AAP polypeptides and the polynucleotides can be used in clinical screens to test for metabolic disease etiology, or to assess the level of risk for these disorders. Tissue samples, especially BAT and WAT, of a patient can be examined for the amount of AAP polypeptide or AAP mRNA. When amounts significantly smaller or larger than normal are found, they are indicative of metabolic disease or risk of metabolic disease. Mutation of AAP can yield altered activity, and a patient with such a mutation may have a metabolic disease or be at risk for a metabolic disease. Finally, determining the amount of expression of AAP in a mammal, in a tissue sample, or in a tissue culture, can be used to discover inducers or repressors of the gene.

High expression of AAP in murine and human BAT and WAT indicates an important role for this protein in regulating adipose tissue physiological function and hence metabolic homeostasis. Modulation of the protein's activity through agonists or antagonists could therefore be useful in treating metabolic disorders and disorders associated with changes in adipose tissue physiological function or mass. Administration of a secreted version of the protein may also be useful to treat such disorders. Furthermore, its striking specificity indicates that AAP may serve as a marker of adipose tissue. Thus, therapeutically, determination of the abundance of AAP mRNA, AAP protein or AAP biological activity may be useful to detect the progression of diseases characterized by perturbations in adipose tissue physiological function or adipose abundance. The efficacy of drugs designed to modulate metabolism, adipose tissue physiological function, or adipose mass may also be monitored by assessing AAP mRNA/protein/activity levels in clinical samples, which would be expected to change relative to alterations in adipose tissue metabolism or growth. Finally, detection of polymorphisms or mutations in the gene or protein in a patient could be predict: (1) the propensity of a person to develop metabolic disease, dysfunctional adipose tissue physiological function, or abnormal adipose amount, (2) disease progression rate/prognosis for said disorders, or (3) responses to therapeutic modalities designed to combat metabolic disease and/or disease characterized by changes in adipose tissue physiological function or mass.

Determination of AAP mRNA, proteins or AAP activity levels in clinical samples may have predictive value for tracking progression of adipogenesis in disorders characterized by changes in this parameter (i.e., obesity, lipoatrophic disorders, lipomas), or in cases in which therapeutic modalities are applied to correct said disorders. Such determinations of AAP may also be useful in screening assays designed to discover drugs or other therapeutic modalites that modulate adipogenesis and/or adipose tissue physiological function.

Variation in the 3′UTR of gene transcripts can modify mRNA stability and hence translation. It is possible that patients suffering from metabolic disorders or disease associated with altered adipose physiological function or amount, or patients destined to have such afflictions, may display disease-specific patterns of AAP variant expression. Thus, determination of the AAP variant profile in clinical samples may be of value in diagnosis or prediction of disease progression, or in assessment of the efficacy of therapeutic modalites to treat said disorders.

Definitions

Unless defined otherwise, all technical and scientific terms have the same meaning as is commonly understood by one of skill in the art to which this invention belongs. The definitions below are presented for clarity.

The recommendations of (Demerec et al., 1966) where these are relevant to genetics are adapted herein. To distinguish between genes (and related nucleic acids) and the proteins that they encode, the abbreviations for genes are indicated by italicized (or underlined) text while abbreviations for the proteins are not italicized. Thus, AAP or AAP refers to the nucleotide sequence that encodes AAP.

“Isolated,” when referred to a molecule, refers to a molecule that has been identified and separated and/or recovered from a component of its natural environment. Contaminant components of its natural environment are materials that interfere with diagnostic or therapeutic use.

### 1. Nucleic Acid-related Definitions

(a) Control Sequences

Control sequences are DNA sequences that enable the expression of an operably-linked coding sequence in a particular host organism. Prokaryotic control sequences include promoters, operator sequences, and ribosome binding sites. Eukaryotic cells utilize promoters, polyadenylation signals, and enhancers.

(b) Operably-linked

Nucleic acid is operably-linked when it is placed into a functional relationship with another nucleic acid sequence. For example, a promoter or enhancer is operably-linked to a coding sequence if it affects the transcription of the sequence, or a ribosome-binding site is operably-linked to a coding sequence if positioned to facilitate translation. Generally, “operably-linked” means that the DNA sequences being linked are contiguous, and, in the case of a secretory leader, contiguous and in reading phase. However, enhancers do not have to be contiguous. Linking can be accomplished by conventional recombinant DNA methods.

(c) Isolated Nucleic Acids

An isolated nucleic acid molecule is purified from the setting in which it is found in nature and is separated from at least one contaminant nucleic acid molecule. Isolated AAP molecules are distinguished from the specific; AAP molecule, as it exists in cells. However, an isolated AAP molecule includes AAP molecules contained in cells that ordinarily express AAP where, for example, the nucleic acid molecule is in a chromosomal location different from that of natural cells.

### 2. Protein-related Definitions

(a) Purified Polypeptide

When the molecule is a purified polypeptide, the polypeptide will be purified (1) to obtain at least 15 residues of N-terminal or internal amino acid sequence using a sequenator, or (2) to homogeneity by SDS-PAGE under non-reducing or reducing conditions using Coomassie blue or silver stain. Isolated polypeptides include those expressed heterologously in genetically-engineered cells or expressed in vitro, since at least one component of the AAP natural environment will not be present. Ordinarily, isolated polypeptides are prepared by at least one purification step.

(b) Active Polypeptide

An active AAP or AAP fragment retains a biological and/or an immunological activity of native or naturally-occurring AAP. Immunological activity refers to the ability to induce the production of an antibody against an antigenic epitope possessed by a native AAP; biological activity refers to a function, either inhibitory or stimulatory, caused by a native AAP that excludes immunological activity. A biological activity of AAP includes, for example, its upregulation in Wnt1-expressing cells.

(c) Abs

Antibody may be single anti-AAP monoclonal Abs (including agonist, antagonist, and neutralizing Abs), anti-AAP antibody compositions with polyepitopic specificity, single chain anti-AAP Abs, and fragments of anti-AAP Abs. A “monoclonal antibody” refers to an antibody obtained from a population of substantially homogeneous Abs, i.e., the individual Abs comprising the population are identical except for naturally-occurring mutations that may be present in minor amounts

(d) Epitope Tags

An epitope tagged polypeptide refers to a chimeric polypeptide fused to a “tag polypeptide”. Such tags provide epitopes against which Abs can be made or are available, but do not interfere with polypeptide activity. To reduce anti-tag antibody reactivity with endogenous epitopes, the tag polypeptide is preferably unique. Suitable tag polypeptides generally have at least six amino acid residues, usually between about 8 and 50 amino acid residues, preferably between 8 and 20 amino acid residues. Examples of epitope tag sequences include HA from Influenza A virus and FLAG.

The novel AAP of the invention include the nucleic acids whose sequences are provided in Tables 1, 2, 3 and 5, or a fragment thereof. The invention also includes mutant or variant AAP, any of whose bases may be changed from the corresponding base shown in Tables 1, 2, 3 and 5 while still encoding a protein that maintains the activities and physiological functions of the AAP fragment, or a fragment of such a nucleic acid. The invention further includes nucleic acids whose sequences are complementary to those just described, including complementary nucleic acid fragments. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as anti-sense binding nucleic acids in therapeutic applications in a subject. In the mutant or variant nucleic acids, and their complements, up to 20% or more of the bases may be so changed.

The invention also includes polypeptides and nucleotides having 80-100%, including 81, 82, 83, 84, 85, 86, 87, 88, 89, 90, 91, 92, 93, 94, 95, 96, 97, 98 and 99%, sequence identity to SEQ ID NOS:1-6, as well as nucleotides encoding any of these polypeptides, and compliments of any of these nucleotides.

The novel AAP of the invention include the protein fragments whose sequences are provided in Tables 4 and 6. The invention also includes an AAP mutant or variant protein, any of whose residues may be changed from the corresponding residue shown in Tables 4 and 6 while still encoding a protein that maintains its native activities and physiological functions, or a functional fragment thereof. In the mutant or variant AAP, up to 20% or more of the residues may be so changed. The invention further encompasses Abs and antibody fragments, such as F_{ab} or (F_{ab})₂, that bind immunospecifically to any of the AAP of the invention.

The sequence shown in Table 1 is one of three cDNAs that encodes human AAP (hAAP). The start and stop codons are indicated by boldface and underlining, respectively.

TABLE 1
hAAP nucleotide sequence cDNA, 1.5 kb
form (SEQ ID NO:1)

| | |
|---|---|
| Ccctgactga gcctgagcct ccctgggaag aggcgcattc | 60 |

ttctctctgc tctctgagct

| | |
|---|---|
| ttccgttgct tccctgccca tctgtgctct cagcagcacc | 120 |

agcaaagttg gcctcaaact

| | |
|---|---|
| tgaacaaagc tgcaggctcc agcgtctttc tgggagctcc | 180 |

gcgggggcag caggcaggcc

| | |
|---|---|
| ccagcctctg aactcagctg gcttgagaag cccagcccaa | 240 |

cccttccagc acccagccgg

| | |
|---|---|
| ccctccgtct cctgagggac gcccctgccc ccgacctgga | 300 |

ggccctcagt ctgggctggg

| | |
|---|---|
| gaatggcgcg ctgaggtccc tccagagccc cttgtcccag | 360 |

cctggagctg cagccgcgca

| | |
|---|---|
| aggcccaggc ccccatggcc cacccggtgc agtccgagtt | 420 |

tccttcagca caggagccag

| | |
|---|---|
| gctccgccgc atccctggac ctgccggaga tggagatact | 480 |

cctcaccaag gcagagaaca

| | |
|---|---|
| aggatgacaa gaccctgaat ctgtccaaga ccctctcggg | 540 |

gcctctggat ctggagcaga

| | |
|---|---|
| acggccaggg cctacccttc aaggccatct ccgaggggca | 600 |

cctggaggcc ccactgcctc

| | |
|---|---|
| ggtccccctc ccgggccagc tcaaggaggg cgtcctccat | 660 |

cgccaccacc tcctatgccc

| | |
|---|---|
| aagaccaaga agcccccaga gattacctca tcctggccgt | 720 |

cgtcgcctgc ttctgccccg

| | |
|---|---|
| tctggcccct caacctcatc cccctcatca tttccatcat | 780 |

gtctcgaagc agcatgcaac

| | |
|---|---|
| agggcaacgt ggacggcgcc cggaggctgg gccgcctggc | 840 |

tcggctgctc agcattaccc

| | |
|---|---|
| tcatcatcat gggcatcgtc attatcatgg tggccgtgac | 900 |

cgtcaacttc acagttcaga

| | |
|---|---|
| agaaataaac ttaagcaggg agctgggcta | 960 |

gcagaggccg gccctggcca

| | |
|---|---|
| tctgacctgc acaccgcggg aggccagggt gctgactgtc | 1020 |

aagcatcccc tggaatgggc

| | |
|---|---|
| acagcacagc cagggcgctc ttccaggcag gcgaggataa | 1080 |

cttgagagtt tcctagggca

| | |
|---|---|
| ccagggacag agctcagagg cccccgaggt gtgtgtagga | 1140 |

ggcggaggcc cgcagagcac

| | |
|---|---|
| agagcaggag aagggcttgg gccctggagg agaaagccat | 1200 |

tctggacacc aggggacctg

| | |
|---|---|
| gacggagggt ccccacagcc cgtgccccac gccgcctgga | 1260 |

ggccagaggg gtcagtggcc

| | |
|---|---|
| ctgctgtccc ggctccatct tggttctagc cgccacctgc | 1320 |

atgaacacag tggcccggct

| | |
|---|---|
| taacgcacta acccagcctc tccctgtgtc ccacagggag | 1380 |

tagcaagacc caccccacac

| | |
|---|---|
| tgccttcacc atctacacca gtgacgccgc tgtgtgtctt | 1440 |

agcatggaaa taaataaacc

| | |
|---|---|
| tgaatgtacg tgctcgcaaa aaaaaaaaaa aaaaaaaaaa | 1500 |

aaaaaaaaaa aaaaaaaaaa

| | |
|---|---|
| aaaaaaaaaa aaaaaaaaa | 1519 |

hAAP is also encoded by a transcript of 1.9 kb (SEQ ID NO:2), presented in Table 2.

TABLE 2
hAAP nucleic acid sequence cDNA, 1.9 kb
form (SEQ ID NO:2)

| | |
|---|---|
| gtgtgcgcac acgcacacac acacacacac acacacacac | 60 |

acagcctccc tgactgagcc

| | |
|---|---|
| tgagcctccc tgggaagagg cgcattcttc tctctgctct | 120 |

ctgagctttc cgttgcttcc

| | |
|---|---|
| ctgcccatct gtgctctcag cagcaccagc aaagttggcc | 180 |

tcaaacttga acaaagctgc

| | |
|---|---|
| aggctccagc gtctttctgg gagctccgcg ggggcagcag | 240 |

gcaggcccca gcctctgaac

| | |
|---|---|
| tcagctggct tgagaagccc agcccaaccc ttccagcacc | 300 |

cagccggccc tccgtctcct

| | |
|---|---|
| gagggacgcc cctgcccccg acctggaggc cctcagtctg | 360 |

ggctggggaa tggcgcgctg

| | |
|---|---|
| aggtccctcc agagcccctt gtcccagcct ggagctgcag | 420 |

ccgcgcaagg cccaggcccc

| | |
|---|---|
| catggcccac ccggtgcagt ccgagtttcc ttcagcacag | 480 |

gagccaggct ccgccgcatc

| | |
|---|---|
| cctggacctg ccggagatgg agatactcct caccaaggca | 540 |

gagaacaagg atgacaagac

| | |
|---|---|
| cctgaatctg tccaagaccc tctcggggcc tctggatctg | 600 |

gagcagaacg gccagggcct

| | |
|---|---|
| acccttcaag gccatctccg aggggcacct ggaggcccca | 660 |

ctgcctcggt ccccctcccg

| | |
|---|---|
| ggccagctca aggagggcgt cctccatcgc caccacctcc | 720 |

tatgcccaag accaagaagc

| | |
|---|---|
| ccccagagat tacctcatcc tggccgtcgt cgcctgcttc | 780 |

tgccccgtct ggcccctcaa

| | |
|---|---|
| cctcatcccc ctcatcattt ccatcatgtc tcgaagcagc | 840 |

atgcaacagg gcaacgtgga

| | |
|---|---|
| cggcgcccgg aggctgggcc gcctggctcg gctgctcagc | 900 |

attaccctca tcatcatggg

| | |
|---|---|
| catcgtcatt atcatggtgg ccgtgaccgt caacttcaca | 960 |

gttcagaaga aata

| | |
|---|---|
| agcagggagc tgggctagca gaggccggcc ctggccatcg | 1020 |

ggagagctct gacctgcaca

| | |
|---|---|
| ccgcgggagg ccagggtgct gactgtcaag catcccctgt | 1080 |

ccccaagttc caaaagcaca

| | |
|---|---|
| gactcaaagg gaaacccccc agtcacccac tgtcagctcc | 1140 |

catctgacaa gaaagcctgg

| | |
|---|---|
| agcgaggaag gacagaaacg aaccgatggt tgagggattg | 1200 |

tcacgggagg aacatgacac

| | |
|---|---|
| ccgaagggac tctaggtgcc ctcggagtgc cacacatgcc | 1260 |

cagaccttct cacacccaca

| | |
|---|---|
| caaataggct ctgccgtgca ctcctacaca cacacacaca | 1320 |

cacacacacg cttgttcaca

| | |
|---|---|
| ctcagaaccc aggacagaca cagccaccgc ttaggggaag | 1380 |

ccactgcaga tgcccctgga

| | |
|---|---|
| atgggcacag cacagccagg gcgctcttcc aggcaggcga | 1440 |

ggataacttg agagtttcct

| | |
|---|---|
| agggcaccag ggacagagct cagaggcccc cgaggtgtgt | 1500 |

gtaggaggcg gaggcccgca

| | |
|---|---|
| gagcacagag caggagaagg gcttgggccc tggaggagaa | 1560 |

agccattctg gacaccaggg

| | |
|---|---|
| gacctggacg gagggtcccc acagcccgtg ccccacgccg | 1620 |

cctggaggcc agaggggtca

| | |
|---|---|
| gtggccctgc tgtcccggct ccatcttggt tctagccgcc | 1680 |

acctgcatga acacagtggc

| | |
|---|---|
| ccggcttaac gcactaaccc agcctctccc tgtgtcccac | 1740 |

agggagtagc aagacccacc

| | |
|---|---|
| ccacactgcc ttcaccatct acaccagtga cgccgctgtg | 1800 |

tgtcttagca tggaaataaa

| | |
|---|---|
| taaacctgaa tgtacgtgct cgccaaaaaa aaaaaaaaaa | 1860 |

aaaaaaaaaa aaaaaaaaaa

| | |
|---|---|
| aaaaaaaaaa aaaaaaaaaa aaaaaaaaa | 1889 |

Finally, hAAP is also encoded by a 2.7 kb transcript.

TABLE 3
hAAP nucleic acid sequence cDNA, 2.7 kb
form (SEQ ID NO:3)

| | |
|---|---|
| ctgctctctg agctttccgt tgcttccctg cccatctgtg | 60 |

ctctcagcag caccagcaaa

| | |
|---|---|
| gttggcctca aacttgaaca aagctgcagg ctccagcgtc | 120 |

tttctgggag ctccgcgggg

| | |
|---|---|
| gcagcaggca ggccccagcc tctgaactca gctggcttga | 180 |

gaagcccagc ccaacccttc

| | |
|---|---|
| cagcacccag ccggccctcc gtctcctgag ggacgcccct | 240 |

gcccccgacc tggaggccct

| | |
|---|---|
| cagtctgggc tggggaatgg cgcgctgagg tccctccaga | 300 |

gccccttgtc ccagcctgga

| | |
|---|---|
| gctgcagccg cgcaaggccc aggcccccat ggcccacccg | 360 |

gtgcagtccg agtttccttc

| | |
|---|---|
| agcacaggag ccaggctccg ccgcatccct ggacctgccg | 420 |

gagatggaga tactcctcac

| | |
|---|---|
| caaggcagag aacaaggatg acaagaccct gaatctgtcc | 480 |

aagaccctct cggggcctct

| | |
|---|---|
| ggatctggag cagaacggcc agggcctacc cttcaaggcc | 540 |

atctccgagg ggcacctgga

| | |
|---|---|
| ggccccactg cctcggtccc cctcccgggc cagctcaagg | 600 |

agggcgtcct ccatcgccac

| | |
|---|---|
| cacctcctat gcccaagacc aagaagcccc cagagattac | 660 |

ctcatcctgg ccgtcgtcgc

| | |
|---|---|
| ctgcttctgc cccgtctggc ccctcaacct catccccctc | 720 |

atcatttcca tcatgtctcg

| | |
|---|---|
| aagcagcatg caacagggca acgtggacgg cgcccggagg | 780 |

ctgggccgcc tggctcggct

| | |
|---|---|
| gctcagcatt accctcatca tcatgggcat cgtcattatc | 840 |

atggtggccg tgaccgtcaa

| | |
|---|---|
| cttcacagtt cagaagaaat aaacttaagc | 900 |

agggagctgg gctagcagag

| | |
|---|---|
| gccatcggga gagctctgac ctgcacaccg cgggaggcca | 960 |

gggtgctgac tgtcaagcat

| | |
|---|---|
| cccctgtccc caagttccaa aagcacagac tcaaagggaa | 1020 |

accccccagt cacccactgt

| | |
|---|---|
| cagctcccat ctgacaagaa agcctggagc gaggaaggaa | 1080 |

agcacagcga acccaatggg

| | |
|---|---|
| taacgtggtt tactctcccg gacgcgtcct gggatctcaa | 1140 |

ccccagcagt ctggcttgtt

| | |
|---|---|
| tctcattccc acaatttcct gggttccacc aagcagcgaa | 1200 |

aactgccagg atgaatgagg

| | |
|---|---|
| aaaaaaccca gccccacaaa cgagacacac gctggcgggg | 1260 |

agagacgcag cagagctcct

| | |
|---|---|
| tcctgtctgt ggactcggag caaagacgtg gggccccatc | 1320 |

ttttgtgttt tcctcaagcg

| | |
|---|---|
| gggaaagaat ggactgtttg catgcttcgt gccacacgcc | 1380 |

cgcggtgatc ccagccaggg

| | |
|---|---|
| ccccgagcgc agaggcggag ctgtgctcag cacaggcctg | 1440 |

ggacctcccc cggcaggcac

| | |
|---|---|
| ctgtgggggg tgcagctccc gggaaggagg caactgcctc | 1500 |

acttaacatc ctccgctgca

| | |
|---|---|
| aggtggtggc gccgagaggc gtgtctgtga agacaggtac | 1560 |

caggatggca ggacccgcac

| | |
|---|---|
| gcctcttccc acacctgtca gcttcggaag catctctcga | 1620 |

ggactctggt cccaggatgt

| | |
|---|---|
| ctcccaggac aagccagtct gcctcttcct cctacttctg | 1680 |

ctgtagcctg ggaccagacc

| | |
|---|---|
| tggccaaggt cagccagcgg ggagggccga ggtctgagct | 1740 |

ctcgtcctgc cgtggccccc

| | |
|---|---|
| gcgatggcct ggggtgcaag cttggggctc cgtctggtgc | 1800 |

tgcttaggta gtttcaggct

| | |
|---|---|
| tccagcccag ccctatgctc tgtgcttggc cctgggcccc | 1860 |

tggccatcca caaacagcag

| | |
|---|---|
| aggccttccc tggaggaggc tttgggacca cccctggggg | 1920 |

gacttcccaa agggtcccag

The polypeptide encoded by SEQ ID NOs:1, 2 and 3 is presented in Table 4.

TABLE 4
hAAP polypeptide sequence (SEQ ID NO:4)

```
Met Ala His Pro Val Gln Ser Glu Phe Pro Ser Ala
1 5 10
Gln Glu Pro Gly Ser Ala Ala Ser Leu Asp Leu Pro
                15 20
Glu Met Glu Ile Leu Leu Thr Lys Ala Glu Asn Lys
25 30 35
Asp Asp Lys Thr Leu Asn Leu Ser Lys Thr Leu Ser
                        40 45
Gly Pro Leu Asp Leu Glu Gln Asn Gly Gln Gly Leu
        50 55 60
Pro Phe Lys Ala Ile Ser Glu Gly His Leu Glu Ala
                                65 70
Pro Leu Pro Arg Ser Pro Ser Arg Ala Ser Ser Arg
                75 80
Arg Ala Ser Ser Ile Ala Thr Thr Ser Tyr Ala Gln
85 90 95
Asp Gln Glu Ala Pro Arg Asp Tyr Leu Ile Leu Ala
                        100 105
Val Val Ala Cys Phe Cys Pro Val Trp Pro Leu Asn
        110 115 120
Leu Ile Pro Leu Ile Ile Ser Ile Met Ser Arg Ser
                                125 130
Ser Met Gln Gln Gly Asn Val Asp Gly Ala Arg Arg
                135 140
Leu Gly Arg Leu Ala Arg Leu Leu Ser Ile Thr Leu
145 150 155
Ile Ile Met Gly Ile Val Ile Ile Met Val Ala Val
                        160 165
Thr Val Asn Phe Thr Val Gln Lys Lys
        170 175
```

The partial-length (5′ open) mouse ortholog of the human AAP sequence (mAAP) is shown in Table 5; the stop codons is indicated by underlining.

TABLE 5
mAAP nucleotide sequence (SEQ ID NO:5)

| | |
|---|---|
| atgccactcc aggtccactg cacctcggtt ctatcgattc | 60 |

gaattcggcc acactggccg

| | |
|---|---|
| gatcctctag agatccctcg acctcgaccc acgcgtccgg | 120 |

gatgaccaag ccctgaatct

| | |
|---|---|
| gtccaagtct ctgtcagggg ctctggacct ggagcagaat | 180 |

ggtcacagcc tgcccttcaa

| | |
|---|---|
| ggtcatctcc gaggggcacc gccagccctc cctttccggc | 240 |

tccccttccc gggtcagctc

| | |
|---|---|
| tcggcgagca tcctccgtca tcaccacatc ctacgcccag | 300 |

gaccaagaag cccccaaaga

| | |
|---|---|
| ttacctggtc cttgccattg cctcttgctt ctgccccgtc | 360 |

tggcccctca acctcatccc

| | |
|---|---|
| ccttatcttt tctatcatgt ctcgaagtag tgtgcagcag | 420 |

ggggacctgg atggggcacg

| | |
|---|---|
| gaggctgggc cgcctggcac ggctactcag catcaccttc | 480 |

atcatcctgg gcatcgttat

| | |
|---|---|
| catcattgtg gctgtcactg tcaatttcac agttccgaag | 540 |

taaccgcgga gctgtttcta

| | |
|---|---|
| gcagaggcag aggcagatgc cggccagctg gagatctccg | 600 |

aacctacatg tccccacaca

| | |
|---|---|
| ttacaaagga caggatggtg ggtgctgagt gcccacctgt | 660 |

ccctaagatc caaaagcaca

| | |
|---|---|
| aacttggagg gaaatctctc catcactcca ttgccagccc | 720 |

acggcagacg ggagggaaaa

| | |
|---|---|
| tctgaaatga agttggctgt ggactcaatc tcagagtcac | 780 |

atctctcggg cttgtttctc

| | |
|---|---|
| atcccacaat tcctgggact tacacgtagc gtaaactgcc | 840 |

agaatgggaa gaaaatcagc

| | |
|---|---|
| cccatagcca agaaagaagc actgggtatc tatgaacgtg | 900 |

agcacatcct ggccgcccgt

| | |
|---|---|
| cttttctatt tttattgagg gggagtaaag cgactgcttg | 960 |

catgctttgt gccgagcatc

| | |
|---|---|
| catggtgatc ccagccacag cccaaggtga atctgtcact | 1020 |

cagcacaggc cctggatacc

| | |
|---|---|
| cactggcaaa gaggcaacgg ccatacggaa atcctccatt | 1080 |

gatgactgac tgaataggga

| | |
|---|---|
| tagtatgaca gacctggtgc tcttgctgaa ggtcacgctg | 1140 |

gaggcttctc taccaaggac

| | |
|---|---|
| tctggatcca gagtatcttc cagaaccaca ggatctgcct | 1200 |

cttgattttc acctgtgatg

| | |
|---|---|
| tagcctgtgg acaccaacct ggaagcccta gaacttgacc | 1260 |

tgtcccaggt cagccatggg

| | |
|---|---|
| ggagggacag ggcttgatct ctctctcttg ccacggactc | 1320 |

tttggaggtc tgggaccccg

| | |
|---|---|
| tcttggtaac ccacacaagt ggctcaggag ccctatacag | 1380 |

ggttttcagg ggaccctaca

| | |
|---|---|
| cagaagagct gaagtagagc ctggcttcag actgttagcc | 1440 |

gagctctaat ggatgctctg

| | |
|---|---|
| tgcctggtcc tgcatccctg gccatccaca gcaacaaagg | 1500 |

ctttccctag aggtagcttt

| | |
|---|---|
| gagactattg gactctcttg tttattcata ggctctgggg | 1560 |

agctgctgca gcatgtaagg

| | |
|---|---|
| gagggggcta gagaattctt aggaaggttt gttatttctt | 1620 |

gatctggagt ccaaggaggg

| | |
|---|---|
| gctcacctga aagggcagtc tggggagtgg ggaccaggac | 1680 |

aggaagcaaa ggcccagtag

| | |
|---|---|
| ccatagcctc tcctgagtag aacacggaaa agaagctaat | 1740 |

ctctgccaaa cagcctcatg

| | |
|---|---|
| cttgttgcat ctctatttct ccactttttt tttttttttt | 1800 |

tttttttttt ggcctggcac

| | |
|---|---|
| aggctgtgcc cacgggggct tcacctgctt gccttgcctg | 1860 |

ctctgagctc catctaccca

| | |
|---|---|
| atgtcatact ccaccaccac caacttcagc tcagacctgg | 1920 |

cagaagggct tcccttggcc

The polypeptide encoded by SEQ ID NO:5 is presented in Table 6.

TABLE 6
mAAP polypeptide sequence (SEQ ID NO:6)

```
Arg Ser Leu Asp Leu Asp Pro Arg Val Arg Asp Asp
1 5 10
Gln Ala Leu Asn Leu Ser Lys Ser Leu Ser Gly Ala
                15 20
Leu Asp Leu Glu Gln Asn Gly His Ser Leu Pro Phe
25 30 35
Lys Val Ile Ser Glu Gly His Arg Gln Pro Ser Leu
                        40 45
Ser Gly Ser Pro Ser Arg Val Ser Ser Arg Arg Ala
        50 55 60
Ser Ser Val Ile Thr Thr Ser Tyr Ala Gln Asp Gln
                                65 70
Glu Ala Pro Lys Asp Tyr Leu Val Leu Ala Ile Ala
                75 80
Ser Cys Phe Cys Pro Val Trp Pro Leu Asn Leu Ile
85 90 95
Pro Leu Ile Phe Ser Ile Met Ser Arg Ser Ser Val
                        100 105
Gln Gln Gly Asp Leu Asp Gly Ala Arg Arg Leu Gly
        110 115 120
Arg Leu Ala Arg Leu Leu Ser Ile Thr Phe Ile Ile
                                125 130
Leu Gly Ile Val Ile Ile Ile Val Ala Val Thr Val
                135 140
Asn Phe Thr Val Pro Lys
145 150
```

AAP Polynucleotides

One aspect of the invention pertains to isolated nucleic acid molecules that encode AAP or biologically-active portions thereof. Also included in the invention are nucleic acid fragments sufficient for use as hybridization probes to identify AAP-encoding nucleic acids (e.g., AAP mRNAs) and fragments for use as polymerase chain reaction (PCR) primers for the amplification and/or mutation of AAP molecules. A “nucleic acid molecule” includes DNA molecules (e.g., cDNA or genomic DNA), RNA molecules (e.g., mRNA), analogs of the DNA or RNA generated using nucleotide analogs, and derivatives, fragments and homologs. The nucleic acid molecule may be single-stranded or double-stranded, but preferably comprises double-stranded DNA.

### 1. Probes

Probes are nucleic acid sequences of variable length, preferably between at least about 10 nucleotides (nt), 100 nt, or many (e.g., 6,000 nt) depending on the specific use. Probes are used to detect identical, similar, or complementary nucleic acid sequences. Longer length probes can be obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies. Probes are substantially purified oligonucleotides that will hybridize under stringent conditions to at least optimally 12, 25, 50, 100, 150, 200, 250, 300, 350 or 400 consecutive sense strand nucleotide sequence of SEQ ID NOS:1, 2, 3 or 5; or an anti-sense strand nucleotide sequence of SEQ ID NOS:1, 2, 3 or 5; or of a naturally occurring mutant of SEQ ID NOS:1, 2, 3 or 5.

The full- or partial length native sequence AAP may be used to “pull out” similar (homologous) sequences (Ausubel et al., 1987; Sambrook, 1989), such as: (1) full-length or fragments of AAP cDNA from a cDNA library from any species (e.g. human, murine, feline, canine, bacterial, viral, retroviral, yeast), (2) from cells or tissues, (3) variants within a species, and (4) homologues and variants from other species. To find related sequences that may encode related genes, the probe may be designed to encode unique sequences or degenerate sequences. Sequences may also be genomic sequences including promoters, enhancer elements and introns of native sequence AAP.

For example, an AAP coding region in another species may be isolated using such probes. A probe of about 40 bases is designed, based on AAP, and made. To detect hybridizations, probes are labeled using, for example, radionuclides such as ³²P or ³⁵S, or enzymatic labels such as alkaline phosphatase coupled to the probe via avidin-biotin systems. Labeled probes are used to detect nucleic acids having a complementary sequence to that of AAP in libraries of cDNA, genomic DNA or mRNA of a desired species.

Such probes can be used as a part of a diagnostic test kit for identifying cells or tissues which mis-express an AAP, such as by measuring a level of AAP in a sample of cells from a subject e.g., detecting AAP mRNA levels or determining whether a genomic AAP has been mutated or deleted.

### 2. Isolated Nucleic Acid

An isolated nucleic acid molecule is separated from other nucleic acid molecules that are present in the natural source of the nucleic acid. Preferably, an isolated nucleic acid is free of sequences that naturally flank the nucleic acid (i.e., sequences located at the 5′- and 3′-termini of the nucleic acid) in the genomic DNA of the organism from which the nucleic acid is derived. For example, in various embodiments, isolated AAP molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an isolated nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOS:1, 2, 3 or 5, or a complement, can be isolated using standard molecular biology techniques and the provided sequence information. Using all or a portion of the nucleic acid sequence of SEQ ID NOS:1, 2, 3 or 5 as a hybridization probe, AAP molecules can be isolated using standard hybridization and cloning techniques (Ausubel et al., 1987; Sambrook, 1989).

PCR amplification techniques can be used to amplify AAP using cDNA, mRNA or alternatively, genomic DNA, as a template and appropriate oligonucleotide primers. Such nucleic acids can be cloned into an appropriate vector and characterized by DNA sequence analysis. Furthermore, oligonucleotides corresponding to AAP sequences can be prepared by standard synthetic techniques, e.g., an automated DNA synthesizer.

### 3. Oligonucleotide

An oligonucleotide comprises a series of linked nucleotide residues, which oligonucleotide has a sufficient number of nucleotide bases to be used in a PCR reaction or other application. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS:1, 2, 3 or 5, or a complement thereof. Oligonucleotides may be chemically synthesized and may also be used as probes.

### 4. Complementary Nucleic Acid Sequences; Binding

In another embodiment, an isolated nucleic acid molecule of the invention comprises a nucleic acid molecule that is a complement of the nucleotide sequence shown in SEQ ID NOS:1, 2, 3 or 5, or a portion of this nucleotide sequence (e.g., a fragment that can be used as a probe or primer or a fragment encoding a biologically-active portion of an AAP). A nucleic acid molecule that is complementary to the nucleotide sequence shown in SEQ ID NOS:1, 2, 3 or 5, is one that is sufficiently complementary to the nucleotide sequence shown in SEQ ID NOS:1, 2, 3 or 5, that it can hydrogen bond with little or no mismatches to the nucleotide sequence shown in SEQ ID NOS:1, 2, 3 or 5, thereby forming a stable duplex.

“Complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Nucleic acid fragments are at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full-length sequence. Fragments may be derived from any contiguous portion of a nucleic acid or amino acid sequence of choice.

### 5. Derivatives and Analogs

Derivatives are nucleic acid sequences or amino acid sequences formed from the native compounds either directly or by modification or partial substitution. Analogs are nucleic acid sequences or amino acid sequences that have a structure similar to, but not identical to, the native compound but differ from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions (Ausubel et al., 1987).

### 6. Homology

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of AAP. Isoforms can be expressed in different tissues of the same organism as a result of, for example, alternative splicing of RNA. Alternatively, different genes can encode isoforms. In the invention, homologous nucleotide sequences include nucleotide sequences encoding for an AAP of species other than humans, including, but not limited to: vertebrates, e.g., frog, mouse, rat, rabbit, dog, cat, cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human AAP. Homologous nucleic acid sequences include those nucleic acid sequences that encode conservative amino acid substitutions (see below) in SEQ ID NOS:4 or 6, as well as a polypeptide possessing AAP biological activity. Various biological activities of the AAP are described below.

### 7. Open Reading Frames

The open reading frame (ORF) of an AAP gene encodes AAP. An ORF is a nucleotide sequence that has a start codon (ATG) and terminates with one of the three “stop” codons (TAA, TAG, or TGA). In this invention, however, an ORF may be any part of a coding sequence that may or may not comprise a start codon and a stop codon. To achieve a unique sequence, preferable AAP ORFs encode at least 50 amino acids.

AAP Polypeptides

### 1. Mature

An AAP can encode a mature AAP. A “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

### 2. Active

An active AAP polypeptide or AAP polypeptide fragment retains a biological and/or an immunological activity similar, but not necessarily identical, to an activity of a naturally-occuring (wild-type) AAP polypeptide of the invention, including mature forms. A particular biological assay, with or without dose dependency, can be used to determine AAP activity. A nucleic acid fragment encoding a biologically-active portion of AAP can be prepared by isolating a portion of SEQ ID NOS:1, 2, 3 or 5 that encodes a polypeptide having an AAP biological activity (the biological activities of the AAP are described below), expressing the encoded portion of AAP (e.g., by recombinant expression in vitro) and assessing the activity of the encoded portion of AAP. Immunological activity refers to the ability to induce the production of an antibody against an antigenic epitope possessed by a native AAP; biological activity refers to a function, either inhibitory or stimulator, caused by a native AAP that excludes immunological activity.

AAP Nucleic Acid Variants and Hybridization

### 1. Variant Polynucleotides, Genes and Recombinant Genes

The invention further encompasses nucleic acid molecules that differ from the nucleotide sequences shown in SEQ ID NOS:1, 2, 3 or 5 due to degeneracy of the genetic code and thus encode the same AAP as that encoded by the nucleotide sequences shown in SEQ ID NO NOS:1, 2, 3 or 5. An isolated nucleic acid molecule of the invention has a nucleotide sequence encoding a protein having an amino acid sequence shown in SEQ ID NOS:4 or 6.

In addition to the AAP sequences shown in SEQ ID NOS:1, 2, 3 or 5, DNA sequence polymorphisms that change the amino acid sequences of the AAP may exist within a population. For example, allelic variation among individuals will exhibit genetic polymorphism in AAP. The terms “gene” and “recombinant gene” refer to nucleic acid molecules comprising an open reading frame (ORF) encoding AAP, preferably a vertebrate AAP. Such natural allelic variations can typically result in 1-5% variance in AAP. Any and all such nucleotide variations and resulting amino acid polymorphisms in the AAP, which are the result of natural allelic variation and that do not alter the functional activity of the AAP are within the scope of the invention.

Moreover, AAP from other species that have a nuclteotide sequence that differs from the sequence of SEQ ID NOS:1, 2, 3 or 5, are contemplated. Nucleic acid molecules corresponding to natural allelic variants and homologues of the AAP cDNAs of the invention can be isolated based on their homology to the AAP of SEQ ID NOS:1, 2, 3 or 5 using cDNA-derived probes to hybridize to homologous AAP sequences under stringent conditions.

“AAP variant polynucleotide” or “AAP variant nucleic acid sequence” means a nucleic acid molecule which encodes an active AAP that (1) has at least about 80% nucleic acid sequence identity with a nucleotide acid sequence encoding a full-length native AAP, (2) a full-length native AAP lacking the signal peptide, (3) an extracellular domain of an AAP, with or without the signal peptide, or (4) any other fragment of a full-length AAP. Ordinarily, an AAP variant polynucleotide will have at least about 80% nucleic acid sequence identity, more preferably at least about 81%, 82%, 83%, 84%, 85%, 86%, 87%, 88%, 89%, 90%, 91%, 92%, 93%, 94%, 95%, 96%, 97%, 98% nucleic acid sequence identity and yet more preferably at least about 99% nucleic acid sequence identity with the nucleic acid sequence encoding a full-length native AAP. An AAP variant polynucleotide may encode full-length native AAP lacking the signal peptide, an extracellular domain of an AAP, with or without the signal sequence, or any other fragment of a full-length AAP. Variants do not encompass the native nucleotide sequence.

Ordinarily, AAP variant polynucleotides are at least about 30 nucleotides in length, often at least about 60, 90, 120, 150, 180, 210, 240, 270, 300, 450, 600 nucleotides in length, more often at least about 900 nucleotides in length, or more.

“Percent (%) nucleic acid sequence identity” with respect to AAP-encoding nucleic acid sequences identified herein is defined as the percentage of nucleotides in a candidate sequence that are identical with the nucleotides in the AAP sequence of interest, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:

% nucleic acid sequence identity=W/Z·100

where

W is the number of nucleotides scored as identical matches by the sequence alignment program's or algorithm's alignment of C and D and

Z is the total number of nucleotides in D.

When the length of nucleic acid sequence C is not equal to the length of nucleic acid sequence D, the % nucleic acid sequence identity of C to D will not equal the % nucleic acid sequence identity of D to C.

### 2. Stringency

Homologs (i.e., nucleic acids encoding AAP derived from species other than human) or other related sequences (e.g., paralogs) can be obtained by low, moderate or high stringency hybridization with all or a portion of the particular human sequence as a probe using methods well known in the art for nucleic acid hybridization and cloning.

The specificity of single stranded DNA to hybridize complementary fragments is determined by the “stringency” of the reaction conditions. Hybridization stringency increases as the propensity to form DNA duplexes decreases. In nucleic acid hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide which decreases DNA duplex stability. In general, the longer the probe, the higher the temperature required for proper annealing. A common approach is to vary the temperature: higher relative temperatures result in more stringent reaction conditions. (Ausubel et al., 1987) provide an excellent explanation of stringency of hybridization reactions.

To hybridize under “stringent conditions” describes hybridization protocols in which nucleotide sequences at least 60% homologous to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

(a) High Stringency

“Stringent hybridization conditions” conditions enable a probe, primer or oligonucleotide to hybridize only to its target sequence. Stringent conditions are sequence-dependent and will differ. Stringent conditions comprise: (1) low ionic strength and high temperature washes (e.g. 15 mM sodium chloride, 1.5 mM sodium citrate, 0.1% sodium dodecyl sulfate at 50° C.); (2) a denaturing agent during hybridization (e.g. 50% (v/v) formamide, 0.1% bovine serum albumin, 0.1% Ficoll, 0.1% polyvinylpyrrolidone, 50 mM sodium phosphate buffer (pH 6.5; 750 mM sodium chloride, 75 mM sodium citrate at 42° C.); or (3) 50% formamide. Washes typically also comprise 5×SSC (0.75 M NaCl, 75 mM sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Preferably, the conditions are such that sequences at least about 65%, 70%, 75%, 85%, 90%, 95%, 98%, or 99% homologous to each other typically remain hybridized to each other. These conditions are presented as examples and are not meant to be limiting.

(b) Moderate Stringency

“Moderately stringent conditions” use washing solutions and hybridization conditions that are less stringent (Sambrook, 1989), such that a polynucleotide will hybridize to the entire, fragments, derivatives or analogs of SEQ ID NOS:1, 2, 3 or 5. One example comprises hybridization in 6×SSC, 5×Denhardt's solution, 0.5% SDS and 100 mg/ml denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions have been described (Ausubel et al., 1987; Kriegler, 1990).

(c) Low Stringency

“Low stringent conditions” use washing solutions and hybridization conditions that are less stringent than those for moderate stringency (Sambrook, 1989), such that a polynucleotide will hybridize to the entire, fragments, derivatives or analogs of SEQ ID NOS:1, 2, 3 or 5. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency, such as those for cross-species hybridizations are well-described (Ausubel et al., 1987; Kriegler, 1990; Shilo and Weinberg, 1981).

### 3. Conservative Mutations

In addition to naturally-occurring allelic variants of AAP, changes can be introduced by mutation into SEQ ID NOS:1, 2, 3 or 5 that incur alterations in the amino acid sequences of the encoded AAP that do not alter AAP function. For example, nucleotide substitutions leading to amino acid substitutions at “non-essential” amino acid residues can be made in the sequence of SEQ ID NOS:4 or 6. A “non-essential” amino acid residue is a residue that can be altered from the wild-type sequences of the AAP without altering biological activity, whereas an “essential” amino acid residue is required for such biological activity. For example, amino acid residues that are conserved among the AAP of the invention are predicted to be particularly non-amenable to alteration. Amino acids for which conservative substitutions can be made are well known in the art.

Useful conservative substitutions are shown in Table A, “Preferred substitutions.” Conservative substitutions whereby an amino acid of one class is replaced with another amino acid of the same type fall within the scope of the invention so long as the substitution does not materially alter the biological activity of the compound. If such substitutions result in a change in biological activity, then more substantial changes, indicated in Table B as exemplary, are introduced and the products screened for AAP polypeptide biological activity.

TABLE A
Preferred substitutions

| Original | Exemplary | Preferred |
|---|---|---|
| residue | substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, Ala, | Ile |
| | Phe | |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, | Leu |
| | Norleucine | |

Non-conservative substitutions that effect (1) the structure of the polypeptide backbone, such as a β-sheet or α-helical conformation, (2) the charge (3) hydrophobicity, or (4) the bulk of the side chain of the target site can modify AAP polypeptide function or immunological identity. Residues are divided into groups based on common side-chain properties as denoted in Table B. Non-conservative substitutions entail exchanging a member of one of these classes for another class. Substitutions may be introduced into conservative substitution sites or more preferably into non-conserved sites.

TABLE B
Amino acid classes

| Class | Amino acids |
|---|---|
| hydrophobic | Norleucine, Met, Ala, Val, Leu, Ile |
| neutral hydrophilic | Cys, Ser, Thr |
| acidic | Asp, Glu |
| basic | Asn, Gln, His, Lys, Arg |
| disrupt chain conformation | Gly, Pro |
| aromatic | Trp, Tyr, Phe |

The variant polypeptides can be made using methods known in the art such as oligonucleotide-mediated (site-directed) mutagenesis, alanine scanning, and PCR mutagenesis. Site-directed mutagenesis (Carter, 1986; Zoller and Smith, 1987), cassette mutagenesis, restriction selection mutagenesis (Wells et al., 1985) or other known techniques can be performed on the cloned DNA to produce the AAP variant DNA (Ausubel et al., 1987; Sambrook, 1989).

In one embodiment, the isolated nucleic acid molecule comprises a nucleotide sequence encoding a protein, wherein the protein comprises an amino acid sequence at least about 45%, preferably 60%, more preferably 70%, 80%, 90%, and most preferably about 95% homologous to SEQ ID NOS:4 or 6.

### 4. Anti-sense Nucleic Acids

Using antisense and sense AAP oligonucleotides can prevent AAP polypeptide expression. These oligonucleotides bind to target nucleic acid sequences, forming duplexes that block transcription or translation of the target sequence by enhancing degradation of the duplexes, terminating prematurely transcription or translation, or by other means.

Antisense or sense oligonucleotides are singe-stranded nucleic acids, either RNA or DNA, which can bind target AAP mRNA (sense) or AAP DNA (antisense) sequences. Anti-sense nucleic acids can be designed according to Watson and Crick or Hoogsteen base pairing rules. The anti-sense nucleic acid molecule can be complementary to the entire coding region of AAP mRNA, but more preferably, to only a portion of the coding or noncoding region of AAP mRNA. For example, the anti-sense oligonucleotide can be complementary to the region surrounding the translation start site of AAP mRNA. Antisense or sense oligonucleotides may comprise a fragment of the AAP DNA coding region of at least about 14 nucleotides, preferably from about 14 to 30 nucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Among others, (Stein and Cohen, 1988; van der Krol et al., 1988b) describe methods to derive antisense or a sense oligonucleotides from a given cDNA sequence.

Examples of modified nucleotides that can be used to generate the anti-sense nucleic acid include: 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the anti-sense nucleic acid can be produced biologically using an expression vector into which a nucleic acid has been sub-cloned in an anti-sense orientation such that the transcribed RNA will be complementary to a target nucleic acid of interest.

To introduce antisense or sense oligonucleotides into target cells (cells containing the target nucleic acid sequence), any gene transfer method may be used. Examples of gene transfer methods include (1) biological, such as gene transfer vectors like Epstein-Barr virus or conjugating the exogenous DNA to a ligand-binding molecule, (2) physical, such as electroporation and injection, and (3) chemical, such as CaPO₄ precipitation and oligonucleotide-lipid complexes.

An antisense or sense oligonucleotide is inserted into a suitable gene transfer retroviral vector. A cell containing the target nucleic acid sequence is contacted with the recombinant retroviral vector, either in vivo or ex vivo. Examples of suitable retroviral vectors include those derived from the murine retrovirus M-MuLV, N2 (a retrovirus derived from M-MuLV), or the double copy vectors designated DCT5A, DCT5B and DCT5C (WO 90/13641, 1990). To achieve sufficient nucleic acid molecule transcription, vector constructs in which the transcription of the anti-sense nucleic acid molecule is controlled by a strong pol II or pol III promoter are preferred. Alternatively, inducible promoters may be preferred when the expression of the construct is desired to be controlled.

To specify target cells in a mixed population of cells cell surface receptors that are specific to the target cells can be exploited. Antisense and sense oligonucleotides are conjugated to a ligand-binding molecule, as described in (WO 91/04753, 1991). Ligands are chosen for receptors that are specific to the target cells. Examples of suitable ligand-binding molecules include cell surface receptors, growth factors, cytokines, or other ligands that bind to cell surface receptors or molecules. Preferably, conjugation of the ligand-binding molecule does not substantially interfere with the ability of the receptors or molecule to bind the ligand-binding molecule conjugate, or block entry of the sense or antisense oligonucleotide or its conjugated version into the cell.

Liposomes efficiently transfer sense or an antisense oligonucleotide to cells (WO 90/10448, 1990). The sense or antisense oligonucleotide-lipid complex is preferably dissociated within the cell by an endogenous lipase.

The anti-sense nucleic acid molecule of the invention may be an α-anomeric nucleic acid molecule. An α-anomeric nucleic acid molecule forms specific double-stranded hybrids with complementary RNA in which, contrary to the usual α-units, the strands run parallel to each other (Gautier et al., 1987). The anti-sense nucleic acid molecule can also comprise a 2′-o-methylribonucleotide (Inoue et al., 1987a) or a chimeric RNA-DNA analogue (Inoue et al., 1987b).

In one embodiment, an anti-sense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes, such as hammerhead ribozymes (Haseloff and Gerlach, 1988) can be used to catalytically cleave AAP mRNA transcripts and thus inhibit translation. A ribozyme specific for an AAP-encoding nucleic acid can be designed based on the nucleotide sequence of an AAP cDNA (i.e., SEQ ID NOS:1, 2, 3 or 5). For example, a derivative of a Tetrahymena L-19 IVS RNA can be constructed in which the nucleotide sequence of the active site is complementary to the nucleotide sequence to be cleaved in an AAP-encoding mRNA (Cech et al., U.S. Pat. No. 5,116,742, 1992; Cech et al., U.S. Pat. No. 4,987,071, 1991). AAP mRNA can also be used to select a catalytic RNA having a specific ribonuclease activity from a pool of RNA molecules (Bartel and Szostak, 1993).

Alternatively, AAP expression can be inhibited by targeting nucleotide sequences complementary to the regulatory region of the AAP (e.g., the AAP promoter and/or enhancers) to form triple helical structures that prevent transcription of the AAP in target cells (Helene, 1991; Helene et al., 1992; Maher, 1992).

Modifications of antisense and sense oligonucleotides can augment their effectiveness. Modified sugar-phosphodiester bonds or other sugar linkages (WO 91/06629, 1991), increase in vivo stability by conferring resistance to endogenous nucleases without disrupting binding specificity to target sequences. Other modifications can increase the affinities of the oligonucleotides for their targets, such as covalently linked organic moieties (WO 90/10448, 1990) or poly-(L)-lysine. Other attachments modify binding specificities of the oligonucleotides for their targets, including metal complexes or intercalating (e.g. ellipticine) and alkylating agents.

For example, the deoxyribose phosphate backbone of the nucleic acids can be modified to generate peptide nucleic acids (Hyrup and Nielsen, 1996). “Peptide nucleic acids” or “PNAs” refer to nucleic acid mimics (e.g., DNA mimics) in that the deoxyribose phosphate backbone is replaced by a pseudopeptide backbone and only the four natural nucleobases are retained. The neutral backbone of PNAs allows for specific hybridization to DNA and RNA under conditions of low ionic strength. The synthesis of PNA oligomers can be performed using standard solid phase peptide synthesis protocols (Hyrup and Nielsen, 1996; Perry-O'Keefe et al., 1996).

PNAs of AAP can be used in therapeutic and diagnostic applications. For example, PNAs can be used as anti-sense or antigene agents for sequence-specific modulation of gene expression by inducing transcription or translation arrest or inhibiting replication. AAP PNAs may also be used in the analysis of single base pair mutations (e.g., PNA directed PCR clamping; as artificial restriction enzymes when used in combination with other enzymes, e.g., S₁ nucleases (Hyrup and Nielsen, 1996); or as probes or primers for DNA sequence and hybridization (Hyrup and Nielsen, 1996; Perry-O'Keefe et al., 1996).

PNAs of AAP can be modified to enhance their stability or cellular uptake. Lipophilic or other helper groups may be attached to PNAs, PNA-DNA dimmers formed, or the use of liposomes or other drug delivery techniques. For example, PNA-DNA chimeras can be generated that may combine the advantageous properties of PNA and DNA. Such chimeras allow DNA recognition enzymes (e.g., RNase H and DNA polymerases) to interact with the DNA portion while the PNA portion provides high binding affinity and specificity. PNA-DNA chimeras can be linked using linkers of appropriate lengths selected in terms of base stacking, number of bonds between the nucleobases, and orientation (Hyrup and Nielsen, 1996). The synthesis of PNA-DNA chimeras can be performed (Finn et al., 1996; Hyrup and Nielsen, 1996). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Finn et al., 1996; Hyrup and Nielsen, 1996). PNA monomers are then coupled in a stepwise manner to produce a chimeric molecule with a 5′ PNA segment and a 3′ DNA segment (Finn et al., 1996). Alternatively, chimeric molecules can be synthesized with a 5′ DNA segment and a 3′ PNA segment (Petersen et al., 1976).

The oligonucleotide may include other appended groups such as peptides (e.g., for targeting host cell receptors in vivo), or agents facilitating transport across the cell membrane (Lemaitre et al., 1987; Letsinger et al., 1989; Tullis, U.S. Pat. No. 4,904,582, 1988) or the blood-brain barrier (e.g., (Pardridge and Schimmel, WO89/10134, 1989)). In addition, oligonucleotides can be modified with hybridization-triggered cleavage agents (van der Krol et al., 1988a) or intercalating agents (Zon, 1988). The oligonucleotide may be conjugated to another molecule, e.g., a peptide, a hybridization triggered cross-linking agent, a transport agent, a hybridization-triggered cleavage agent, and the like.

AAP Polypeptides

One aspect of the invention pertains to isolated AAP, and biologically-active portions derivatives, fragments, analogs or homologs thereof. Also provided are polypeptide fragments suitable for use as immunogens to raise anti-AAP Abs. In one embodiment, native AAP can be isolated from cells or tissue sources by an appropriate purification scheme using standard protein purification techniques. In another embodiment, AAP are produced by recombinant DNA techniques. Alternative to recombinant expression, an AAP or polypeptide can be synthesized chemically using standard peptide synthesis techniques.

### 1. Polypeptides

An AAP polypeptide includes the amino acid sequence of AAP which sequences are provided in SEQ ID NOS:4 or 6. The invention also includes a mutant or variant protein any of which residues may be changed from the corresponding residues shown in SEQ ID NOS:4 or 6, while still encoding a protein that maintains AAP activities and physiological functions, or a functional fragment thereof.

### 2. Variant AAP Polypeptides

In general, an AAP variant that preserves AAP-like function and includes any variant in which residues at a particular position in the sequence have been substituted by other amino acids, and further includes the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

“AAP polypeptide variant” means an active AAP polypeptide having at least: (1) about 80% amino acid sequence identity with a full-length native sequence AAP polypeptide sequence, (2) an AAP polypeptide sequence lacking the signal peptide, (3) an extracellular domain of an AAP polypeptide, with or without the signal peptide, or (4) any other fragment of a full-length AAP polypeptide sequence. For example, AAP polypeptide variants include AAP polypeptides wherein one or more amino acid residues are added or deleted at the N- or C-terminus of the full-length native amino acid sequence. An AAP polypeptide variant will have at least about 80% amino acid sequence identity, preferably at least about 81% amino acid sequence identity, more preferably at least about 82%, 83%, 84%, 85%, 86%, 87%, 88%, 89%, 90%, 91%, 92%, 93%, 94%, 95%, 96%, 97%, 98% amino acid sequence identity and most preferably at least about 99% amino acid sequence identity with a full-length native sequence AAP polypeptide sequence. An AAP polypeptide variant may have a sequence lacking the signal peptide, an extracellular domain of an AAP polypeptide, with or without the signal peptide, or any other fragment of a full-length AAP polypeptide sequence. Ordinarily, AAP variant polypeptides are at least about 10 amino acids in length, often at least about 20 amino acids in length, more often at least about 30, 40, 50, 60, 70, 80, 90, 100, 150, 200, or 300 amino acids in length, or more.

“Percent (%) amino acid sequence identity” is defined as the percentage of amino acid residues that are identical with amino acid residues in the disclosed AAP polypeptide sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

% amino acid sequence identity=X/Y·100

where

X is the number of amino acid residues scored as identical matches by the sequence alignment program's or algorithm's alignment of A and B and

Y is the total number of amino acid residues in B.

If the length of amino acid sequence A is not equal to the length of amino acid sequence B, the % amino acid sequence identity of A to B will not equal the % amino acid sequence identity of B to A.

### 3. Isolated/Purified Polypeptides

An “isolated” or “purified” polypeptide, protein or biologically active fragment is separated and/or recovered from a component of its natural environment. Contaminant components include materials that would typically interfere with diagnostic or therapeutic uses for the polypeptide, and may include enzymes, hormones, and other proteinaceous or non-proteinaceous materials. Preferably, the polypeptide is purified to a sufficient degree to obtain at least 15 residues of N-terminal or internal amino acid sequence. To be substantially isolated, preparations having less than 30% by dry weight of non-AAP contaminating material (contaminants), more preferably less than 20%, 10% and most preferably less than 5% contaminants. An isolated, recombinantly-produced AAP or biologically active portion is preferably substantially free of culture medium, i.e., culture medium represents less than 20%, more preferably less than about 10%, and most preferably less than about 5% of the volume of the AAP preparation. Examples of contaminants include cell debris, culture media, and substances used and produced during in vitro synthesis of AAP.

### 4. Biologically Active

Biologically active portions of AAP include peptides comprising amino acid sequences sufficiently homologous to or derived from the amino acid sequences of AAP (SEQ ID NOS:4 or 6) that include fewer amino acids than the full-length AAP, and exhibit at least one activity of an AAP. Biologically active portions comprise a domain or motif with at least one activity of native AAP. A biologically active portion of an AAP can be a polypeptide that is, for example, 10, 25, 50, 100 or more amino acid residues in length. Other biologically active portions, in which other regions of the protein are deleted, can be prepared by recombinant techniques and evaluated for one or more of the functional activities of a native AAP.

Biologically active portions of AAP may have an amino acid sequence shown in SEQ ID NOS:4 or 6, or substantially homologous to SEQ ID NOS:4 or 6, and retains the functional activity of the protein of SEQ ID NOS:4 or 6, yet differs in amino acid sequence due to natural allelic variation or mutagenesis. Other biologically active AAP may comprise an amino acid sequence at least 45% homologous to the amino acid sequence of SEQ ID NOS:4 or 6, and retains the functional activity of native AAP.

### 5. Determining Homology Between Two or More Sequences

“AAP variant” means an active AAP having at least: (1) about 80% amino acid sequence identity with a full-length native sequence AAP sequence, (2) an AAP sequence lacking the signal peptide, (3) an extracellular domain of an AAP, with or without the signal peptide, or (4) any other fragment of a fall-length AAP sequence. For example, AAP variants include AAP wherein one or more amino acid residues are added or deleted at the N- or C-terminus of the full-length native amino acid sequence. An AAP variant will have at least about 80% amino acid sequence identity, preferably at least about 81% amino acid sequence identity, more preferably at least about 82%, 83%, 84%, 85%, 86%, 87%, 88%, 89%, 90%, 91%, 92%, 93%, 94%, 95%, 96%, 97%, 98% amino acid sequence identity and most preferably at least about 99% amino acid sequence identity with a full-length native sequence AAP sequence. An AAP variant may have a sequence lacking the signal peptide, an extracellular domain of an AAP, with or without the signal peptide, or any other fragment of a full-length AAP sequence. Ordinarily, AAP variant polypeptides are at least about 10 amino acids in length, often at least about 20 amino acids in length, more often at least about 30, 40, 50, 60, 70, 80, 90, 100, 150, 200, or 300 amino acids in length, or more.

“Percent (%) amino acid sequence identity” is defined as the percentage of amino acid residues that are identical with amino acid residues in the disclosed AAP sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

% amino acid sequence identity=X/Y·100

where

X is the number of amino acid residues scored as identical matches by the sequence alignment program's or algorithm's alignment of A and B and

Y is the total number of amino acid residues in B.

If the length of amino acid sequence A is not equal to the length of amino acid sequence B, the % amino acid sequence identity of A to B will not equal the % amino acid sequence identity of B to A.

### 6. Chimeric and Fusion Proteins

Fusion polypeptides are useful in expression studies, cell-localization, bioassays, and AAP purification. An AAP “chimeric protein” or “fusion protein” comprises AAP fused to a non-AAP polypeptide. A non-AAP polypeptide is not substantially homologous to AAP (SEQ ID NOS:4 or 6). An AAP fusion protein may include any portion to the entire AAP, including any number of the biologically active portions. AAP may be fused to the C-terminus of the GST (glutathione S-transferase) sequences. Such fusion proteins facilitate the purification of recombinant AAP. In certain host cells, (e.g. mammalian), heterologous signal sequences fusions may ameliorate AAP expression and/or secretion. Additional exemplary fusions are presented in Table C.

Other fusion partners can adapt AAP therapeutically. Fusions with members of the immunoglobulin (Ig) protein family are useful in therapies that inhibit AAP ligand or substrate interactions, consequently suppressing AAP-mediated signal transduction in vivo. AAP-Ig fusion polypeptides can also be used as immunogens to produce anti-AAP Abs in a subject, to purify AAP ligands, and to screen for molecules that inhibit interactions of AAP with other molecules.

Fusion proteins can be easily created using recombinant methods. A nucleic acid encoding AAP can be fused in-frame with a non-AAP encoding nucleic acid, to the AAP NH₂— or COO— terminus, or internally. Fusion genes may also be synthesized by conventional techniques, including automated DNA synthesizers. PCR amplification using anchor primers that give rise to complementary overhangs between two consecutive gene fragments that can subsequently be annealed and reamplified to generate a chimeric gene sequence (Ausubel et al., 1987) is also useful. Many vectors are commercially available that facilitate subcloning AAP in-frame to a fusion moiety.

TABLE C
Useful non-AAP fusion polypeptides

| Reporter | in vitro | in vivo | Notes | Reference |
|---|---|---|---|---|
| Human growth | Radioimmuno- | none | Expensive, | (Selden et al., |
| hormone (hGH) | assay | | insensitive, | 1986) |
| | | | narrow linear | |
| | | | range. | |
| β-glucu- | Colorimetric, | colorimetric | sensitive, | (Gallagher, |
| ronidase (GUS) | fluorescent, | (histo-chemical | broad linear | 1992) |
| | or chemi- | staining with | range, non- | |
| | luminescent | X-gluc) | iostopic. | |
| Green | Fluorescent | fluorescent | can be used in | (Chalfie et al., |
| fluorescent | | | live cells; | 1994) |
| protein (GFP) | | | resists photo- | |
| and related | | | bleaching | |
| molecules (RFP, | | | | |
| BFP, AAP, etc.) | | | | |
| Luciferase | bioluminsecent | Bio- | protein is | (de Wet et al., |
| (firefly) | | luminescent | unstable, | 1987) |
| | | | difficult to | |
| | | | reproduce, | |
| | | | signal is brief | |
| Chloramphenicoal | Chromato- | none | Expensive | (Gorman et al., |
| acetyltransferase | graphy, | | radioactive | 1982) |
| (CAT) | differential | | substrates, | |
| | extraction, | | time- | |
| | fluorescent, | | consuming, | |
| | or immunoassay | | insensitive, | |
| | | | narrow linear | |
| | | | range | |
| β-galacto-sidase | colorimetric, | colorimetric | sensitive, | (Alam and |
| | fluorescence, | (histochemical | broad linear | Cook, 1990) |
| | chemi- | staining with | range; some | |
| | luminscence | X-gal), bio- | cells have high | |
| | | luminescent in | endogenous | |
| | | live cells | activity | |
| Secrete alkaline | colorimetric, | none | Chemi- | (Berger et al., |
| phosphatase | bioluminescent, | | luminscence | 1988) |
| (SEAP) | chemi- | | assay is | |
| | luminescent | | sensitive and | |
| | | | broad linear | |
| | | | range, some | |
| | | | cells have | |
| | | | endogenouse | |
| | | | alkaline | |
| | | | phosphatase | |
| | | | activity | |
| Tat from HIV | Mediates | Mediates | Exploits amino | (Frankel et al., |
| | delivery into | delivery into | acid residues | U.S. Pat. No. |
| | cytoplasm and | cytoplasm and | of HIV tat | 5,804,604, |
| | nuclei | nuclei | protein. | 1998) |

Therapeutic Applications of AAP

### 1. Agonists and Antagonists

“Antagonist” includes any molecule that partially or fully blocks, inhibits, or neutralizes a biological activity of endogenous AAP. Similarly, “agonist” includes any molecule that mimics or enhances a biological activity of endogenous AAP. Molecules that can act as agonists or antagonists include Abs or antibody fragments, fragments or variants of endogenous AAP, peptides, antisense oligonucleotides, small organic molecules, etc.

### 2. Identifying Antagonists and Agonists

To assay for antagonists, AAP is added to, or expressed in, a cell along with the compound to be screened for a particular activity. If the compound inhibits the activity of interest in the presence of the AAP, that compound is an antagonist to the AAP; if AAP activity is enhanced, the compound is an agonist.

(a) Specific Examples of Potential Antagonists and Agonist

Any molecule that alters AAP cellular effects is a candidate antagonist or agonist. Screening techniques well known to those skilled in the art can identify these molecules. Examples of antagonists and agonists include: (1) small organic and inorganic compounds, (2) small peptides, (3) Abs and derivatives, (4) polypeptides closely related to AAP, (5) antisense DNA and RNA, (6) ribozymes, (7) triple DNA helices and (8) nucleic acid aptamers.

Small molecules that bind to the AAP active site or other relevant part of the polypeptide and inhibit the biological activity of the AAP are antagonists. Examples of small molecule antagonists include small peptides, peptide-like molecules, preferably soluble, and synthetic non-peptidyl organic or inorganic compounds. These same molecules, if they enhance AAP activity, are examples of agonists.

Almost any antibody that affects AAP's function is a candidate antagonist, and occasionally, agonist. Examples of antibody antagonists include polyclonal, monoclonal, single-chain, anti-idiotypic, chimeric Abs, or humanized versions of such Abs or fragments. Abs may be from any species in which an immune response can be raised. Humanized Abs are also contemplated.

Alternatively, a potential antagonist or agonist may be a closely related protein, for example, a mutated form of the AAP that recognizes an AAP-interacting protein but imparts no effect, thereby competitively inhibiting AAP action. Alternatively, a mutated AAP may be constitutively activated and may act as an agonist.

Antisense RNA or DNA constructs can be effective antagonists. Antisense RNA or DNA molecules block function by inhibiting translation by hybridizing to targeted mRNA. Antisense technology can be used to control gene expression through triple-helix formation or antisense DNA or RNA, both of which depend on polynucleotide binding to DNA or RNA. For example, the 5′ coding portion of the AAP sequence is used to design an antisense RNA oligonucleotide of from about 10 to 40 base pairs in length. A DNA oligonucleotide is designed to be complementary to a region of the gene involved in transcription (triple helix) (Beal and Dervan, 1991; Cooney et al., 1988; Lee et al., 1979), thereby preventing transcription and the production of the AAP. The antisense RNA oligonucleotide hybridizes to the mRNA in vivo and blocks translation of the mRNA molecule into the AAP (antisense) (Cohen, 1989; Okano et al., 1991). These oligonucleotides can also be delivered to cells such that the antisense RNA or DNA may be expressed in vivo to inhibit production of the AAP. When antisense DNA is used, oligodeoxyribonucleotides derived from the translation-initiation site, e.g., between about −10 and +10 positions of the target gene nucleotide sequence, are preferred.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques (WO 97/33551, 1997; Rossi, 1994).

To inhibit transcription, triple-helix nucleic acids that are single-stranded and comprise deoxynucleotides are useful antagonists. These oligonucleotides are designed such that triple-helix formation via Hoogsteen base-pairing rules is promoted, generally requiring stretches of purines or pyrimidines (WO 97/33551, 1997).

Aptamers are short oligonucleotide sequences that can be used to recognize and specifically bind almost any molecule. The systematic evolution of ligands by exponential enrichment (SELEX) process (Ausubel et al., 1987; Ellington and Szostak, 1990; Tuerk and Gold, 1990) is powerful and can be used to find such aptamers. Aptamers have many diagnostic and clinical uses; almost any use in which an antibody has been used clinically or diagnostically, aptamers too may be used. In addition, aptamers are cheaper to make once they have been identified, and can be easily applied in a variety of formats, including administration in pharmaceutical compositions, bioassays and diagnostic tests (Jayasena, 1999).

Anti-AAP Abs

The invention encompasses Abs and antibody fragments, such as F_{ab} or (F_{ab})₂, that bind immunospecifically to any AAP epitopes.

“Antibody” (Ab) comprises single Abs directed against AAP (anti-AAP Ab; including agonist, antagonist, and neutralizing Abs), anti-AAP Ab compositions with poly-epitope specificity, single chain anti-AAP Abs, and fragments of anti-AAP Abs. A “monoclonal antibody” is obtained from a population of substantially homogeneous Abs, i.e., the individual Abs comprising the population are identical except for possible naturally-occurring mutations that may be present in minor amounts. Exemplary Abs include polyclonal (pAb), monoclonal (mAb), humanized, bi-specific (bsAb), and heteroconjugate Abs.

### 1. Polyclonal Abs (pAbs)

Polyclonal Abs can be raised in a mammalian host, for example, by one or more injections of an immunogen and, if desired, an adjuvant. Typically, the immunogen and/or adjuvant are injected in the mammal by multiple subcutaneous or intraperitoneal injections. The immunogen may include AAP or a fusion protein. Examples of adjuvants include Freund's complete and monophosphoryl Lipid A synthetic-trehalose dicorynomycolate (MPL-TDM). To improve the immune response, an immunogen may be conjugated to a protein that is immunogenic in the host, such as keyhole limpet hemocyanin (KLH), serum albumin, bovine thyroglobulin, and soybean trypsin inhibitor. Protocols for antibody production are described (Ausubel et al., 1987; Harlow and Lane, 1988). Alternatively, pAbs maybe made in chickens, producing IgY molecules (Schade et al., 1996).

### 2. Monoclonal Abs (mAbs)

Anti-AAP mAbs may be prepared using hybridoma methods (Milstein and Cuello, 1983). Hybridoma methods comprise at least four steps: (1) immunizing a host, or lymphocytes from a host; (2) harvesting the mAb secreting (or potentially secreting) lymphocytes, (3) fusing the lymphocytes to immortalized cells, and (4) selecting those cells that secrete the desired (anti-AAP) mAb.

A mouse, rat, guinea pig, hamster, or other appropriate host is immunized to elicit lymphocytes that produce or are capable of producing Abs that will specifically bind to the immunogen. Alternatively, the lymphocytes may be immunized in vitro. If human cells are desired, peripheral blood lymphocytes (PBLs) are generally used; however, spleen cells or lymphocytes from other mammalian sources are preferred. The immunogen typically includes AAP or a fusion protein.

The lymphocytes are then fused with an immortalized cell line to form hybridoma cells, facilitated by a fusing agent such as polyethylene glycol (Goding, 1996). Rodent, bovine, or human myeloma cells immortalized by transformation may be used, or rat or mouse myeloma cell lines. Because pure populations of hybridoma cells and not unfused immortalized cells are preferred, the cells after fusion are grown in a suitable medium that contains one or more substances that inhibit the growth or survival of unfused, immortalized cells. A common technique uses parental cells that lack the enzyme hypoxanthine guanine phosphoribosyl transferase (HGPRT or HPRT). In this case, hypoxanthine, aminopterin and thymidine are added to the medium (HAT medium) to prevent the growth of HGPRT-deficient cells while permitting hybridomas to grow.

Preferred immortalized cells fuse efficiently; can be isolated from mixed populations by selecting in a medium such as HAT; and support stable and high-level expression of antibody after fusion. Preferred immortalized cell lines are murine myeloma lines, available from the American Type Culture Collection (Manassas, Va.). Human myeloma and mouse-human heteromyeloma cell lines also have been described for the production of human mAbs (Kozbor et al., 1984; Schook, 1987).

Because hybridoma cells secrete antibody extracellularly, the culture media can be assayed for the presence of mAbs directed against AAP (anti-AAP mAbs). Immunoprecipitation or in vitro binding assays, such as radio immunoassay (RIA) or enzyme-linked immunoabsorbent assay (ELISA), measure the binding specificity of mAbs (Harlow and Lane, 1988; Harlow and Lane, 1999), including Scatchard analysis (Munson and Rodbard, 1980).

Anti-AAP mAb secreting hybridoma cells may be isolated as single clones by limiting dilution procedures and sub-cultured (Goding, 1996). Suitable culture media include Dulbecco's Modified Eagle's Medium, RPMI-1640, or if desired, a protein-free or -reduced or serum-free medium (e.g., Ultra DOMA PF or HL-1; Biowhittaker; Walkersville, Md.). The hybridoma cells may also be grown in vivo as ascites.

The mAbs may be isolated or purified from the culture medium or ascites fluid by conventional Ig purification procedures such as protein A-Sepharose, hydroxylapatite chromatography, gel electrophoresis, dialysis, ammonium sulfate precipitation or affinity chromatography (Harlow and Lane, 1988; Harlow and Lane, 1999).

The mAbs may also be made by recombinant methods (U.S. Pat. No. 4,166,452, 1979). DNA encoding anti-AAP mAbs can be readily isolated and sequenced using conventional procedures, e.g., using oligonucleotide probes that specifically bind to murine heavy and light antibody chain genes, to probe preferably DNA isolated from anti-AAP-secreting mAb hybridoma cell lines. Once isolated, the isolated DNA fragments are sub-cloned into expression vectors that are then transfected into host cells such as simian COS-7 cells, Chinese hamster ovary (CHO) cells, or myeloma cells that do not otherwise produce Ig protein, to express mAbs. The isolated DNA fragments can be modified, for example, by substituting the coding sequence for human heavy and light chain constant domains in place of the homologous murine sequences (U.S. Pat. No. 4,816,567, 1989; Morrison et al., 1987), or by fusing the Ig coding sequence to all or part of the coding sequence for a non-Ig polypeptide. Such a non-Ig polypeptide can be substituted for the constant domains of an antibody, or can be substituted for the variable domains of one antigen-combining site to create a chimeric bivalent antibody.

### 3. Monovalent Abs

The Abs may be monovalent Abs that consequently do not cross-link with each other. For example, one method involves recombinant expression of Ig light chain and modified heavy chain. Heavy chain truncations at any point in the F_{c} region will prevent heavy chain cross-linking. Alternatively, the relevant cysteine residues are substituted with another amino acid residue or are deleted, preventing crosslinking. In vitro methods are also suitable for preparing monovalent Abs. Abs can be digested to produce fragments, such as F_{ab} fragments (Harlow and Lane, 1988; Harlow and Lane, 1999) that will not cross link.

### 4. Humanized and Human Abs

Anti-AAP Abs may further comprise humanized or human Abs. Humanized forms of non-human Abs are chimeric Igs, Ig chains or fragments (such as Fᵥ, F_{ab}, F_{ab′}, F_{(ab′)2} or other antigen-binding subsequences of Abs) that contain minimal sequence derived from non-human Ig.

Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody (Jones et al., 1986; Riechmann et al., 1988; Verhoeyen et al., 1988). Such “humanized” Abs are chimeric Abs (U.S. Pat. No. 4,816,567, 1989), wherein substantially less than an intact human variable domain has been substituted by the corresponding sequence from a non-human species. In practice, humanized Abs are typically human Abs in which some CDR residues and possibly some FR residues are substituted by residues from analogous sites in rodent Abs. Humanized Abs include human Igs (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fᵥ framework residues of the human Ig. Humanized Abs may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human Ig consensus sequence. The humanized antibody optimally also comprises at least a portion of an Ig constant region (F_{c}), typically that of a human Ig (Jones et al., 1986; Presta, 1992; Riechmann et al., 1988).

Human Abs can also be produced using various techniques, including phage display libraries (Hoogenboom et al., 1991; Marks et al., 1991) and the preparation of human mAbs (Boerner et al., 1991; Reisfeld and Sell, 1985). Similarly, introducing human Ig genes into transgenic animals in which the endogenous Ig genes have been partially or completely inactivated can be exploited to synthesize human Abs. Upon challenge, human antibody production is observed, which closely resembles that seen in humans in all respects, including gene rearrangement, assembly, and antibody repertoire (U.S. Pat. No. 5,545,807, 1996; U.S. Pat. No. 5,545,806, 1996; U.S. Pat. No. 5,569,825, 1996; U.S. Pat. No. 5,633,425, 1997; U.S. Pat. No. 5,661,016, 1997; U.S. Pat. No. 5,625,126, 1997; Fishwild et al., 1996; Lonberg and Huszar, 1995; Lonberg et al., 1994; Marks et al., 1992).

### 5. Bi-specific mAbs

Bi-specific Abs are monoclonal, preferably human or humanized, that have binding specificities for at least two different antigens. For example, a binding specificity is AAP; the other is for any antigen of choice, preferably a cell-surface protein or receptor or receptor subunit.

Traditionally, the recombinant production of bi-specific Abs is based on the co-expression of two Ig heavy-chain/light-chain pairs, where the two heavy chains have different specificities (Milstein and Cuello, 1983). Because of the random assortment of Ig heavy and light chains, the resulting hybridomas (quadromas) produce a potential mixture of ten different antibody molecules, of which only one has the desired bi-specific structure. The desired antibody can be purified using affinity chromatography or other techniques (WO 93/08829, 1993; Traunecker et al., 1991).

To manufacture a bi-specific antibody (Suresh et al., 1986), variable domains with the desired antibody-antigen combining sites are fused to Ig constant domain sequences. The fusion is preferably with an Ig heavy-chain constant domain, comprising at least part of the hinge, CH2, and CH3 regions. Preferably, the first heavy-chain constant region (CH1) containing the site necessary for light-chain binding is in at least one of the fusions. Nucleotide sequences encoding the Ig heavy-chain fusions and, if desired, the Ig light chain, are inserted into separate expression vectors and are co-transfected into a suitable host organism.

The interface between a pair of antibody molecules can be engineered to maximize the percentage of heterodimers that are recovered from recombinant cell culture (WO 96/27011, 1996). The preferred interface comprises at least part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This mechanism increases the yield of the heterodimer over unwanted end products such as homodimers.

Bi-specific Abs can be prepared as full length Abs or antibody fragments (e.g. F_{(ab′)2} bi-specific Abs). One technique to generate bi-specific Abs exploits chemical linkage. Intact Abs can be proteolytically cleaved to generate F_{(ab′)2} fragments (Brennan et al., 1985). Fragments are reduced with a dithiol complexing agent, such as sodium arsenite, to stabilize vicinal dithiols and prevent intermolecular disulfide formation. The generated F_{ab′} fragments are then converted to thionitrobenzoate (TNB) derivatives. One of the F_{ab′}-TNB derivatives is then reconverted to the F_{ab′}-thiol by reduction with mercaptoethylamine and is mixed with an equimolar amount of the other F_{ab′}-TNB derivative to form the bi-specific antibody. The produced bi-specific Abs can be used as agents for the selective immobilization of enzymes.

F_{ab′} fragments may be directly recovered from E. coli and chemically coupled to form bi-specific Abs. For example, fully humanized bi-specific F_{(ab′)2} Abs can be produced (Shalaby et al., 1992). Each F_{ab′} fragment is separately secreted from E. coli and directly coupled chemically in vitro, forming the bi-specific antibody.

Various techniques for making and isolating bi-specific antibody fragments directly from recombinant cell culture have also been described. For example, leucine zipper motifs can be exploited (Kostelny et al., 1992). Peptides from the Fos and Jun proteins are linked to the F_{ab′} portions of two different Abs by gene fusion. The antibody homodimers are reduced at the hinge region to form monomers and then re-oxidized to form antibody heterodimers. This method can also produce antibody homodimers. The “diabody” technology (Holliger et al., 1993) provides an alternative method to generate bi-specific antibody fragments. The fragments comprise a heavy-chain variable domain (V_{H}) connected to a light-chain variable domain (V_{L}) by a linker that is too short to allow pairing between the two domains on the same chain. The V_{H} and V_{L} domains of one fragment are forced to pair with the complementary V_{L} and V_{H} domains of another fragment, forming two antigen-binding sites. Another strategy for making bi-specific antibody fragments is the use of single-chain Fᵥ (sFᵥ) dimers (Gruber et al., 1994). Abs with more than two valencies are also contemplated, such as tri-specific Abs (Tutt et al., 1991).

Exemplary bi-specific Abs may bind to two different epitopes on a given AAP. Alternatively, cellular defense mechanisms can be restricted to a particular cell expressing the particular AAP: an anti-AAP arm may be combined with an arm that binds to a leukocyte triggering molecule, such as a T-cell receptor molecule (e.g. CD2, CD3, CD28, or B7), or to F_{c} receptors for IgG (F_{c}γR), such as F_{c}γRI (CD64), F_{c}γRII (CD32) and F_{c}γRIII (CD16). Bi-specific Abs may also be used to target cytotoxic agents to cells that express a particular AAP. These Abs possess an AAP-binding arm and an arm that binds a cytotoxic agent or a radionuclide chelator.

### 6. Heteroconjugate Abs

Heteroconjugate Abs, consisting of two covalently joined Abs, have been proposed to target immune system cells to unwanted cells (U.S. Pat. No. 4,676,980, 1987) and for treatment of human immunodeficiency virus (HIV) infection (WO 91/00360, 1991; WO 92/20373, 1992). Abs prepared in vitro using synthetic protein chemistry methods, including those involving cross-linking agents, are contemplated. For example, immunotoxins may be constructed using a disulfide exchange reaction or by forming a thioether bond. Examples of suitable reagents include iminothiolate and methyl-4-mercaptobutyrimidate (U.S. Pat. No. 4,676,980, 1987).

### 7. Immunoconjugates

Immunoconjugates may comprise an antibody conjugated to a cytotoxic agent such as a chemotherapeutic agent, toxin (e.g., an enzymatically active toxin or fragment of bacterial, fungal, plant, or animal origin), or a radioactive isotope (i.e., a radioconjugate).

Useful enzymatically-active toxins and fragments include Diphtheria A chain, non-binding active fragments of Diphtheria toxin, exotoxin A chain from Pseudomonas aeruginosa, ricin A chain, abrin A chain, modeccin A chain, α-sarcin, Aleurites fordii proteins, Dianthin proteins, Phytolaca americana proteins, Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated Abs, such as ²¹²Bi, ¹³¹I, ¹³¹In, ⁹⁰Y, and ¹⁸⁶Re.

Conjugates of the antibody and cytotoxic agent are made using a variety of bi-functional protein-coupling agents, such as N-succinimidyl-3-(2-pyridyldithiol) propionate (SPDP), iminothiolane (IT), bi-functional derivatives of imidoesters (such as dimethyl adipimidate HCl), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared (Vitetta et al., 1987). ¹⁴C-labeled 1 -isothiocyanatobenzyl-3-methyldiethylene triaminepentaacetic acid (MX-DTPA) is an exemplary chelating agent for conjugating radionuclide to antibody (WO 94/11026, 1994).

In another embodiment, the antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a streptavidin “ligand” (e.g., biotin) that is conjugated to a cytotoxic agent (e.g., a radionuclide).

### 8. Effector Function Engineering

The antibody can be modified to enhance its effectiveness in treating a disease, such as cancer. For example, cysteine residue(s) may be introduced into the F_{c} region, thereby allowing interchain disulfide bond formation in this region. Such homodimeric Abs may have improved internalization capability and/or increased complement-mediated cell killing and antibody-dependent cellular cytotoxicity (ADCC) (Caron et al., 1992; Shopes, 1992). Homodimeric Abs with enhanced anti-tumor activity can be prepared using hetero-bifunctional cross-linkers (Wolff et al., 1993). Alternatively, an antibody engineered with dual F_{c} regions may have enhanced complement lysis (Stevenson et al., 1989).

### 9. Immunoliposomes

Liposomes containing the antibody may also be formulated (U.S. Pat. No. 4,485,045, 1984; U.S. Pat. No. 4,544,545, 1985; U.S. Pat. No. 5,013,556, 1991; Eppstein et al., 1985; Hwang et al., 1980). Useful liposomes can be generated by a reverse-phase evaporation method with a lipid composition comprising phosphatidylcholine, cholesterol, and PEG-derivatized phosphatidylethanolamine (PEG-PE). Such preparations are extruded through filters of defined pore size to yield liposomes with a desired diameter. F_{ab′} fragments of the antibody can be conjugated to the liposomes (Martin and Papahadjopoulos, 1982) via a disulfide-interchange reaction. A chemotherapeutic agent, such as Doxorubicin, may also be contained in the liposome (Gabizon et al., 1989). Other useful liposomes with different compositions are contemplated.

### 10. Diagnostic Applications of Abs Directed Against AAP

Anti-AAP Abs can be used to localize and/or quantitate AAP (e.g., for use in measuring levels of AAP within tissue samples or for use in diagnostic methods, etc.). Anti-AAP epitope Abs can be utilized as pharmacologically active compounds.

Anti-AAP Abs can be used to isolate AAP by standard techniques, such as immunoaffinity chromatography or immunoprecipitation. These approaches facilitate purifying endogenous AAP antigen-containing polypeptides from cells and tissues. These approaches, as well as others, can be used to detect AAP in a sample to evaluate the abundance and pattern of expression of the antigenic protein. Anti-AAP Abs can be used to monitor protein levels in tissues as part of a clinical testing procedure; for example, to determine the efficacy of a given treatment regimen. Coupling the antibody to a detectable substance (label) allows detection of Ab-antigen complexes. Classes of labels include fluorescent, luminescent, bioluminescent, and radioactive materials, enzymes and prosthetic groups. Useful labels include horseradish peroxidase, alkaline phosphatase, β-galactosidase, acetylcholinesterase, streptavidin/biotin, avidin/biotin, umbelliferone, fluorescein, fluorescein isothiocyanate, rhodamine, dichlorotriazinylamine fluorescein, dansyl chloride, phycoerythrin, luminol, luciferase, luciferin, aequorin, and ¹²⁵I, ¹³¹I, ³⁵S or ³H.

### 11. Antibody Therapeutics

Abs of the invention, including polyclonal, monoclonal, humanized and fully human Abs, can be used therapeutically. Such agents will generally be employed to treat or prevent a disease or pathology in a subject. An antibody preparation, preferably one having high antigen specificity and affinity generally mediates an effect by binding the target epitope(s). Generally, administration of such Abs may mediate one of two effects: (1) the antibody may prevent ligand binding, eliminating endogenous ligand binding and subsequent signal transduction, or (2) the antibody elicits a physiological result by binding an effector site on the target molecule, initiating signal transduction.

A therapeutically effective amount of an antibody relates generally to the amount needed to achieve a therapeutic objective, epitope binding affinity, administration rate, and depletion rate of the antibody from a subject. Common ranges for therapeutically effective doses may be, as a nonlimiting example, from about 0.1 mg/kg body weight to about 50 mg/kg body weight. Dosing frequencies may range, for example, from twice daily to once a week.

### 12. Pharmaceutical Compositions of Abs

Anti-AAP Abs, as well as other AAP interacting molecules (such as aptamers) identified in other assays, can be administered in pharmaceutical compositions to treat various disorders. Principles and considerations involved in preparing such compositions, as well as guidance in the choice of components can be found in (de Boer, 1994; Gennaro, 2000; Lee, 1990).

Abs that are internalized are preferred when whole Abs are used as inhibitors. Liposomes may also be used as a delivery vehicle for intracellular introduction. Where antibody fragments are used, the smallest inhibitory fragment that specifically binds to the epitope is preferred. For example, peptide molecules can be designed that bind a preferred epitope based on the variable-region sequences of a useful antibody. Such peptides can be synthesized chemically and/or produced by recombinant DNA technology (Marasco et al., 1993). Formulations may also contain more than one active compound for a particular treatment, preferably those with activities that do not adversely affect each other. The composition may comprise an agent that enhances function, such as a cytotoxic agent, cytokine, chemotherapeutic agent, or growth-inhibitory agent.

The active ingredients can also be entrapped in microcapsules prepared by coacervation techniques or by interfacial polymerization; for example, hydroxymethylcellulose or gelatin-microcapsules and poly-(methylmethacrylate) microcapsules, respectively, in colloidal drug delivery systems (for example, liposomes, albumin microspheres, micro emulsions, nano-particles, and nanocapsules) or in macroemulsions.

The formulations to be used for in vivo administration are highly preferred to be sterile. This is readily accomplished by filtration through sterile filtration membranes or any of a number of techniques.

Sustained-release preparations may also be prepared, such as semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (Boswell and Scribner, U.S. Pat. No. 3,773,919, 1973), copolymers of L-glutamic acid and γ ethyl-L-glutamate, non-degradable ethylene-vinyl acetate, degradable lactic acid-glycolic acid copolymers such as injectable microspheres composed of lactic acid-glycolic acid copolymer, and poly-D-(−)-3-hydroxybutyric acid. While polymers such as ethylene-vinyl acetate and lactic acid-glycolic acid enable release of molecules for over 100 days, certain hydrogels release proteins for shorter time periods and may be preferred.

AAP Recombinant Expression Vectors and Host Cells

Vectors are tools used to shuttle DNA between host cells or as a means to express a nucleotide sequence. Some vectors function only in prokaryotes, while others function in both prokaryotes and eukaryotes, enabling large-scale DNA preparation from prokaryotes for expression in eukaryotes. Inserting the DNA of interest, such as AAP nucleotide sequence or a fragment, is accomplished by ligation techniques and/or mating protocols well known to the skilled artisan. Such DNA is inserted such that its integration does not disrupt any necessary components of the vector. In the case of vectors that are used to express the inserted DNA protein, the introduced DNA is operably-linked to the vector elements that govern its transcription and translation.

Vectors can be divided into two general classes: Cloning vectors are replicating plasmid or phage with regions that are non-essential for propagation in an appropriate host cell, and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA. In expression vectors, the introduced DNA is operably-linked to elements, such as promoters, that signal to the host cell to transcribe the inserted DNA. Some promoters are exceptionally useful, such as inducible promoters that control gene transcription in response to specific factors. Operably-linking AAP or anti-sense construct to an inducible promoter can control the expression of AAP or fragments, or anti-sense constructs. Examples of classic inducible promoters include those that are responsive to α-interferon, heat-shock, heavy metal ions, and steroids such as glucocorticoids (Kaufman, 1990) and tetracycline. Other desirable inducible promoters include those that are not endogenous to the cells in which the construct is being introduced, but, however, is responsive in those cells when the induction agent is exogenously supplied.

Vectors have many difference manifestations. A “plasmid” is a circular double stranded DNA molecule into which additional DNA segments can be introduced. Viral vectors can accept additional DNA segments into the viral genome. Certain vectors are capable of autonomous replication in a host cell (e.g., episomal mammalian vectors or bacterial vectors having a bacterial origin of replication). Other vectors (e.g., non-episomal mammalian vectors) are integrated into the genome of a host cell upon introduction into the host cell, and thereby are replicated along with the host genome. In general, useful expression vectors are often plasmids. However, other forms of expression vectors, such as viral vectors (e.g., replication defective retroviruses, adenoviruses and adeno-associated viruses) are contemplated.

Recombinant expression vectors that comprise AAP (or fragments) regulate AAP transcription by exploiting one or more host cell-responsive (or that can be manipulated in vitro) regulatory sequences that is operably-linked to AAP. “Operably-linked” indicates that a nucleotide sequence of interest is linked to regulatory sequences such that expression of the nucleotide sequence is achieved.

Vectors can be introduced in a variety of organisms and/or cells (Table D). Alternatively, the vectors can be transcribed and translated in vitro, for example using T7 promoter regulatory sequences and T7 polymerase.

TABLE D
Examples of hosts for cloning or expression

| | | Sources and |
|---|---|---|
| Organisms | Examples | References* |
| Prokaryotes | | |
| | E. coli | |
| | K 12 strain MM294 | ATCC 31,446 |
| | X1776 | ATCC 31,537 |
| | W3110 | ATCC 27,325 |
| | K5 772 | ATCC 53,635 |
| | Enterobacter | |
| | Erwinia | |
| | Klebsiella | |
| Enterobacteriaceae | Proteus | |
| | Salmonella (S. tyhpimurium) | |
| | Serratia (S. marcescans) | |
| | Shigella | |
| | Bacilli (B. subtilis and | |
| | B. licheniformis) | |
| | Pseudomonas | |
| | (P. aeruginosa) | |
| | Streptomyces | |
| Eukaryotes | | |
| Yeasts | Saccharomyces cerevisiae | |
| | Schizosaccharomyces pombe | |
| | Kluyveromyces | (Fleer et al., 1991) |
| | K. lactis MW98-8C, | (de Louvencourt et |
| | CBS683, CBS4574 | al., 1983) |
| | K. fragilis | ATCC 12,424 |
| | K. bulgaricus | ATCC 16,045 |
| | K. wickeramii | ATCC 24,178 |
| | K. waltii | ATCC 56,500 |
| | K. drosophilarum | ATCC 36,906 |
| | K. thermotolerans | |
| | K. marxianus; yarrowia | (EPO 402226, 1990) |
| | Pichia pastoris | (Sreekrishna et al., |
| | Candida | 1988) |
| | Trichoderma reesia | |
| | Neurospora crassa | (Case et al., 1979) |
| | Torulopsis | |
| | Rhodotorula | |
| | Schwanniomyces | |
| | (S. occidentalis) | |
| Filamentous Fungi | Neurospora | |
| | Penicillium | |
| | Tolypocladium | (WO 91/00357, |
| | | 1991) |
| | Aspergillus | (Kelly and Hynes, |
| | (A. nidulans and | 1985; Tilburn et al., |
| | A. niger) | 1983; Yelton et al., |
| | | 1984) |
| Invertebrate cells | Drosophila S2 | |
| | Spodoptera Sf9 | |
| Vertebrate cells | Chinese Hamster Ovary | |
| | (CHO) | |
| | simian COS | |
| | COS-7 | ATCC CRL 1651 |
| | HEK 293 | |

*Unreferenced cells are generally available from American Type Culture Collection (Manassas, VA).

Vector choice is dictated by the organism or cells being used and the desired fate of the vector. Vectors may replicate once in the target cells, or may be “suicide” vectors. In general, vectors comprise signal sequences, origins of replication, marker genes, enhancer elements, promoters, and transcription termination sequences. The choice of these elements depends on the organisms in which the vector will be used and are easily determined. Some of these elements may be conditional, such as an inducible or conditional promoter that is turned “on” when conditions are appropriate. Examples of inducible promoters include those that are tissue-specific, which relegate expression to certain cell types, steroid-responsive, or heat-shock reactive. Some bacterial repression systems, such as the lac operon, have been exploited in mammalian cells and transgenic animals (Fieck et al., 1992; Wyborski et al., 1996; Wyborski and Short, 1991). Vectors often use a selectable marker to facilitate identifying those cells that have incorporated the vector. Many selectable markers are well known in the art for the use with prokaryotes, usually antibiotic-resistance genes or the use of autotrophy and auxotrophy mutants.

Using antisense and sense AAP oligonucleotides can prevent AAP polypeptide expression. These oligonucleotides bind to target nucleic acid sequences, forming duplexes that block transcription or translation of the target sequence by enhancing degradation of the duplexes, terminating prematurely transcription or translation, or by other means.

Antisense or sense oligonucleotides are singe-stranded nucleic acids, either RNA or DNA, which can bind target AAP mRNA (sense) or AAP DNA (antisense) sequences. According to the present invention, antisense or sense oligonucleotides comprise a fragment of the AAP DNA coding region of at least about 14 nucleotides, preferably from about 14 to 30 nucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Among others, (Stein and Cohen, 1988; van der Krol et al., 1988b) describe methods to derive antisense or a sense oligonucleotides from a given cDNA sequence.

Modifications of antisense and sense oligonucleotides can augment their effectiveness. Modified sugar-phosphodiester bonds or other sugar linkages (WO 91/06629, 1991), increase in vivo stability by conferring resistance to endogenous nucleases without disrupting binding specificity to target sequences. Other modifications can increase the affinities of the oligonucleotides for their targets, such as covalently linked organic moieties (WO 90/10448, 1990) or poly-(L)-lysine. Other attachments modify binding specificities of the oligonucleotides for their targets, including metal complexes or intercalating (e.g. ellipticine) and alkylating agents.

To introduce antisense or sense oligonucleotides into target cells (cells containing the target nucleic acid sequence), any gene transfer method may be used and are well known to those of skill in the art. Examples of gene transfer methods include 1) biological, such as gene transfer vectors like Epstein-Barr virus or conjugating the exogenous DNA to a ligand-binding molecule (WO 91/04753, 1991), 2) physical, such as electroporation, and 3) chemical, such as CaPO₄ precipitation and oligonucleotide-lipid complexes (WO 90/10448, 1990).

The terms “host cell” and “recombinant host cell” are used interchangeably. Such terms refer not only to a particular subject cell but also to the progeny or potential progeny of such a cell. Because certain modifications may occur in succeeding generations due to either mutation or environmental influences, such progeny may not, in fact, be identical to the parent cell, but are still included within the scope of the term.

Methods of eukaryotic cell transfection and prokaryotic cell transformation are well known in the art. The choice of host cell will dictate the preferred technique for introducing the nucleic acid of interest. Table E, which is not meant to be limiting, summarizes many of the known techniques in the art. Introduction of nucleic acids into an organism may also be done with ex vivo techniques that use an in vitro method of transfection, as well as established genetic techniques, if any, for that particular organism.

TABLE E
Methods to introduce nucleic acid into cells

| Cells | Methods | References | Notes |
|---|---|---|---|
| Prokaryotes | Calcium chloride | (Cohen et al., 1972; | |
| (bacteria) | | Hanahan, 1983; Mandel | |
| | | and Higa, 1970) | |
| | Electroporation | (Shigekawa and Dower, | |
| | | 1988) | |
| Eukaryotes | | | |
| Mammalian | Calcium | N-(2- | Cells may be |
| cells | phosphate | Hydroxyethyl)piperazine- | “shocked” with |
| | transfection | N′-(2-ethanesulfonic acid | glycerol or |
| | | (HEPES) buffered saline | dimethylsulfoxide |
| | | solution (Chen and | (DMSO) to increase |
| | | Okayama, 1988; Graham | transfection |
| | | and van der Eb, 1973; | efficiency |
| | | Wigler et al., 1978) | (Ausubel et al., |
| | | BES (N,N-bis(2- | 1987). |
| | | hydroxyethyl)-2- | |
| | | aminoethanesulfonic acid) | |
| | | buffered solution (Ishiura | |
| | | et al., 1982) | |
| | Diethylaminoethyl | (Fujita et al., 1986; Lopata | Most useful for |
| | (DEAE)-Dextran | et al., 1984; Selden et al., | transient, but not |
| | transfection | 1986) | stable, |
| | | | transfections. |
| | | | Chloroquine can be |
| | | | used to increase |
| | | | efficiency. |
| | Electroporation | (Neumann et al., 1982; | Especially useful |
| | | Potter, 1988; Potter et al., | for hard-to-transfect |
| | | 1984; Wong and Neumann, | lymphocytes. |
| | | 1982) | |
| | Cationic lipid | (Elroy-Stein and Moss, | Applicable to both |
| | reagent | 1990; Felgner et al., 1987; | in vivo and in vitro |
| | transfection | Rose et al, 1991; Whitt et | transfection. |
| | | al., 1990) | |
| | Retroviral | Production exemplified by | Lengthy process, |
| | | (Cepko et al., 1984; Miller | many packaging |
| | | and Buttimore, 1986; Pear | lines available at |
| | | et al., 1993) | ATCC. Applicable |
| | | Infection in vitro and in | to both in vivo and |
| | | vivo: (Austin and Cepko, | in vitro transfection. |
| | | 1990; Bodine et al., 1991; | |
| | | Fekete and Cepko, 1993; | |
| | | Lemischka et al., 1986; | |
| | | Turner et al., 1990; | |
| | | Williams et al., 1984) | |
| | Polybrene | (Chaney et al., 1986; | |
| | | Kawai and Nishizawa, | |
| | | 1984) | |
| | Microinjection | (Capecchi, 1980) | Can be used to |
| | | | establish cell lines |
| | | | carrying integrated |
| | | | copies of AAP |
| | | | DNA sequences. |
| | Protoplast fusion | (Rassoulzadegan et al., | |
| | | 1982; Sandri-Goldin et al., | |
| | | 1981; Schaffner, 1980) | |
| Insect cells | Baculovirus | (Luckow, 1991; Miller, | Useful for in vitro |
| (in vitro) | systems | 1988; O'Reilly et al., 1992) | production of |
| | | | proteins with |
| | | | eukaryotic |
| | | | modifications. |
| Yeast | Electroporation | (Becker and Guarente, | |
| | | 1991) | |
| | Lithium acetate | (Gietz et al., 1998; Ito et | |
| | | al., 1983) | |
| | Spheroplast fusion | (Beggs, 1978; Hinnen et | Laborious, can |
| | | al., 1978) | produce aneuploids. |
| Plant cells | Agrobacterium | (Bechtold and Pelletier, | |
| (general | transformation | 1998; Escudero and Hohn, | |
| reference: | | 1997; Hansen and Chilton, | |
| Hansen and | | 1999; Touraev and al., | |
| Wright, | | 1997) | |
| 1999)) | Biolistics | (Finer et al., 1999; Hansen | |
| | (microprojectiles) | and Chilton, 1999; Shillito, | |
| | | 1999) | |
| | Electroporation | (Fromm et al., 1985; Ou- | |
| | (protoplasts) | Lee et al., 1986; Rhodes et | |
| | | al., 1988; Saunders et al., 1989) | |
| | | May be combined with | |
| | | liposomes (Trick and al., | |
| | | 1997) | |
| | Polyethylene | (Shillito, 1999) | |
| | glycol (PEG) | | |
| | treatment | | |
| | Liposomes | May be combined with | |
| | | electroporation (Trick and | |
| | | al., 1997) | |
| | in planta | (Leduc and al., 1996; Zhou | |
| | microinjection | and al., 1983) | |
| | Seed imbibition | (Trick and al., 1997) | |
| | Laser beam | (Hoffman, 1996) | |
| | Silicon carbide | (Thompson and al., 1995) | |
| | whiskers | | |

Vectors often use a selectable marker to facilitate identifying those cells that have incorporated the vector. Many selectable markers are well known in the art for the use with prokaryotes, usually antibiotic-resistance genes or the use of autotrophy and auxotrophy mutants. Table F lists often-used selectable markers for mammalian cell transfection.

TABLE F
Useful selectable markers for eukaryote cell transfection

| Selectable | | | |
|---|---|---|---|
| Marker | Selection | Action | Reference |
| Adenosine | Media includes 9-β-D- | Conversion of Xyl-A | (Kaufman et |
| deaminase | xylofuranosyl adenine | to Xyl-ATP, which | al., 1986) |
| (ADA) | (Xyl-A) | incorporates into | |
| | | nucleic acids, killing | |
| | | cells. ADA detoxifies | |
| Dihydro- | Methotrexate (MTX) | MTX competitive | (Simonsen |
| folate | and dialyzed serum | inhibitor of DHFR. In | and |
| reductase | (purine-free media) | absence of exogenous | Levinson, |
| (DHFR) | | purines, cells require | 1983) |
| | | DHFR, a necessary | |
| | | enzyme in purine | |
| | | biosynthesis. | |
| Amino- | G418 | G418, an | (Southern |
| glycoside | | aminoglycoside | and Berg, |
| phospho- | | detoxified by APH, | 1982) |
| transferase | | interferes with | |
| (“APH”, | | ribosomal function | |
| “neo”, | | and consequently, | |
| “G418”) | | translation. | |
| Hygro- | hygromycin-B | Hygromycin-B, and | (Palmer et |
| mycin-B- | | aminocyclitol | al., 1987) |
| phospho- | | detoxified by HPH, | |
| transferase | | disrupts protein | |
| (HPH) | | translocation and | |
| | | promotes | |
| | | mistranslation. | |
| Thymidine | Forward selection | Forward: | (Littlefield, |
| kinase | (TK+): Media (HAT) | Aminopterin forces | 1964) |
| (TK) | incorporates | cells to synthesze | |
| | aminopterin. | dTTP from thymidine, | |
| | Reverse selection | a pathway requiring | |
| | (TK−): Media | TK. | |
| | incorporates 5- | Reverse: TK | |
| | bromodeoxyuridine | phosphorylates BrdU, | |
| | (BrdU). | which incorporates | |
| | | into nucleic acids, | |
| | | killing cells. | |

A host cell of the invention, such as a prokaryotic or eukaryotic host cell in culture can be used to produce AAP. Accordingly, the invention provides methods for producing AAP using the host cells of the invention. In one embodiment, the method comprises culturing the host cell of the invention (into which a recombinant expression vector encoding AAP has been introduced) in a suitable medium, such that AAP is produced. In another embodiment, the method further comprises isolating AAP from the medium or the host cell.

Transgenic AAP Animals

Transgenic animals are useful for studying the function and/or activity of AAP and for identifying and/or evaluating modulators of AAP activity. “Transgenic animals” are non-human animals, preferably mammals, more preferably rodents such as rats or mice, in which one or more of the cells include a transgene. Other transgenic animals include primates, sheep, dogs, cows, goats, chickens, amphibians, etc. A “transgene” is exogenous DNA that is integrated into the genome of a cell from which a transgenic animal develops and remains in the genome of the mature animal. Transgenes preferably direct the expression of an encoded gene product in one or more cell types or tissues of the transgenic animal with the purpose of preventing expression of a naturally encoded gene product in one or more cell types or tissues (a “knockout” transgenic animal), or serving as a marker or indicator of an integration, chromosomal location, or region of recombination (e.g. cre/loxP mice). A “homologous recombinant animal” is a non-human animal, such as a rodent, in which endogenous AAP has been altered by an exogenous DNA molecule that recombines homologously with endogenous AAP in a (e.g. embryonic) cell prior to development the animal. Host cells with exogenous AAP can be used to produce non-human transgenic animals, such as fertilized oocytes or embryonic stem cells into which NAP-coding sequences have been introduced. Such host cells can then be used to create non-human transgenic animals or homologous recombinant animals.

### 1. Approaches to Transgenic Animal Production

A transgenic animal can be created by introducing AAP into the male pronuclei of a fertilized oocyte (e.g., by microinjection, retroviral infection) and allowing the oocyte to develop in a pseudopregnant female foster animal (pffa). The AAP cDNA sequences (SEQ ID NO:1, 2, 3 or 5) can be introduced as a transgene into the genome of a non-human animal. Alternatively, a homologue of AAP, such as the naturally-occuring variant of AAP, can be used as a transgene. Intronic sequences and polyadenylation signals can also be included in the transgene to increase transgene expression. Tissue-specific regulatory sequences can be operably-linked to the AAP transgene to direct expression of AAP to particular cells. Methods for generating transgenic animals via embryo manipulation and microinjection, particularly animals such as mice, have become conventional in the art, e.g. (Evans et al., U.S. Pat. No. 4,870,009, 1989; Hogan Ser. No., 08/79,693,843, 1994; Leder and Stewart, U.S. Pat. No. 4,736,866, 1988; Wagner and Hoppe, U.S. Pat. No. 4,873,191, 1989). Other non-mice transgenic animals may be made by similar methods. A transgenic founder animal, which can be used to breed additional transgenic animals, can be identified based upon the presence of the transgene in its genome and/or expression of the transgene mRNA in tissues or cells of the animals. Transgenic (e.g. AAP) animals can be bred to other transgenic animals carrying other transgenes.

### 2. Vectors for Transgenic Animal Production

To create a homologous recombinant animal, a vector containing at least a portion of AAP into which a deletion, addition or substitution has been introduced to thereby alter, e.g., functionally disrupt, AAP. AAP can be a murine gene (SEQ ID NO:5), or other AAP homologue, such as the human homolog (SEQ ID NOS:1, 2 or 3). In one approach, a knockout vector functionally disrupts the endogenous AAP gene upon homologous recombination, and thus a non-functional AAP protein, if any, is expressed.

Alternatively, the vector can be designed such that, upon homologous recombination, the endogenous AAP is mutated or otherwise altered but still encodes functional protein (e.g., the upstream regulatory region can be altered to thereby alter the expression of endogenous AAP). In this type of homologous recombination vector, the altered portion of the AAP is flanked at its 5′- and 3′-termini by additional nucleic acid of the AAP to allow for homologous recombination to occur between the exogenous AAP carried by the vector and an endogenous AAP in an embryonic stem cell. The additional flanking AAP nucleic acid is sufficient to engender homologous recombination with endogenous AAP. Typically, several kilobases of flanking DNA (both at the 5′- and 3′-termini) are included in the vector (Thomas and Capecchi, 1987). The vector is then introduced into an embryonic stem cell line (e.g., by electroporation), and cells in which the introduced AAP has homologously-recombined with the endogenous AAP are selected (Li et al., 1992).

### 3. Introduction of AAP Transgene Cells During Development

Selected cells are then injected into a blastocyst of an animal (e.g., a mouse) to form aggregation chimeras (Bradley, 1987). A chimeric embryo can then be implanted into a suitable pffa and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described (Berns et al., WO 93/04169, 1993; Bradley, 1991; Kucherlapati et al., WO 91/01140, 1991; Le Mouellic and Brullet, WO 90/11354, 1990).

Alternatively, transgenic animals that contain selected systems that allow for regulated expression of the transgene can be produced. An example of such a system is the cre/loxP recombinase system of bacteriophage P1 (Lakso et al., 1992). Another recombinase system is the FLP recombinase system of Saccharomyces cerevisiae (O'Gorman et al., 1991). If a cre/loxP recombinase system is used to regulate expression of the transgene, animals containing transgenes encoding both the Cre recombinase and a selected protein are required. Such animals can be produced as “double” transgenic animals, by mating an animal containing a transgene encoding a selected protein to another containing a transgene encoding a recombinase.

Clones of transgenic animals can also be produced (Wilmut et al., 1997). In brief, a cell from a transgenic animal can be isolated and induced to exit the growth cycle and enter G₀ phase. The quiescent cell can then be fused to an enucleated oocyte from an animal of the same species from which the quiescent cell is isolated. The reconstructed oocyte is then cultured to develop to a morula or blastocyte and then transferred to a pffa. The offspring borne of this female foster animal will be a clone of the “parent” transgenic animal.

Pharmaceutical Compositions

The AAP nucleic acid molecules, AAP polypeptides, and anti-AAP Abs (active compounds) of the invention, and derivatives, fragments, analogs and homologs thereof, can be incorporated into pharmaceutical compositions. Such compositions typically comprise the nucleic acid molecule, protein, or antibody and a pharmaceutically acceptable carrier. A “pharmaceutically acceptable carrier” includes any and all solvents, dispersion media, coatings, antibacterial and antifungal agents, isotonic and absorption delaying agents, and the like, compatible with pharmaceutical administration (Gennaro, 2000). Preferred examples of such carriers or diluents include, but are not limited to, water, saline, Finger's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

### 1. General Considerations

A pharmaceutical composition of the invention is formulated to be compatible with its intended route of administration, including intravenous, intradermal, subcutaneous, oral (e.g., inhalation), transdermal (i.e., topical), transmucosal, and rectal administration. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampules, disposable syringes or multiple dose vials made of glass or plastic.

### 2. Injectable Formulations

Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a solvent or dispersion medium containing, for example, water, ethanol, polyol (such as glycerol, propylene glycol, and liquid polyethylene glycol), and suitable mixtures. Proper fluidity can be maintained, for example, by using a coating such as lecithin, by maintaining the required particle size in the case of dispersion and by using surfactants. Various antibacterial and antifungal agents, for example, parabens, chlorobutanol, phenol, ascorbic acid, and thimerosal, can contain microorganism contamination. Isotonic agents, for example, sugars, polyalcohols such as manitol, sorbitol, and sodium chloride can be included in the composition. Compositions that can delay absorption include agents such as aluminum monostearate and gelatin.

Sterile injectable solutions can be prepared by incorporating the active compound (e.g., an AAP or anti-AAP antibody) in the required amount in an appropriate solvent with one or a combination of ingredients as required, followed by sterilization. Generally, dispersions are prepared by incorporating the active compound into a sterile vehicle that contains a basic dispersion medium, and the other required ingredients. Sterile powders for the preparation of sterile injectable solutions, methods of preparation include vacuum drying and freeze-drying that yield a powder containing the active ingredient and any desired ingredient from a sterile solutions.

### 3. Oral Compositions

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

### 4. Compositions for Inhalation

For administration by inhalation, the compounds are delivered as an aerosol spray from a nebulizer or a pressurized container that contains a suitable propellant, e.g., a gas such as carbon dioxide.

### 5. Systemic Administration

Systemic administration can also be transmucosal or transdermal. For transmucosal or transdermal administration, penetrants that can permeate the target barrier(s) are selected. Transmucosal penetrants include, detergents, bile salts, and fusidic acid derivatives. Nasal sprays or suppositories can be used for transmucosal administration. For transdermal administration, the active compounds are formulated into ointments, salves, gels, or creams.

The compounds can also be prepared in the form of suppositories (e.g., with bases such as cocoa butter and other glycerides) or retention enemas for rectal delivery.

### 6. Carriers

In one embodiment, the active compounds are prepared with carriers that protect the compound against rapid elimination from the body, such as a controlled release formulation, including implants and microencapsulated delivery systems. Biodegradable or biocompatible polymers can be used, such as ethylene vinyl acetate, polyanhydrides, polyglycolic acid, collagen, polyorthoesters, and polylactic acid. Such materials can be obtained commercially from ALZA Corporation (Mountain View, Calif.) and NOVA Pharmaceuticals, Inc. (Lake Elsinore, Calif.), or prepared by one of skill in the art. Liposomal suspensions can also be used as pharmaceutically acceptable carriers. These can be prepared according to methods known to those skilled in the art, such as in (Eppstein et al., U.S. Pat. No. 4,522,811, 1985).

### 7. Unit Dosage

Oral formulations or parenteral compositions in unit dosage form can be created to facilitate administration and dosage uniformity. Unit dosage form refers to physically discrete units suited as single dosages for the subject to be treated, containing a therapeutically effective quantity of active compound in association with the required pharmaceutical carrier. The specification for the unit dosage forms of the invention are dictated by, and directly dependent on, the unique characteristics of the active compound and the particular desired therapeutic effect, and the inherent limitations of compounding the active compound.

### 8. Gene Therapy Compositions

The nucleic acid molecules of the invention can be inserted into vectors and used as gene therapy vectors. Gene therapy vectors can be delivered to a subject by, for example, intravenous injection, local administration (Nabel and Nabel, U.S. Pat. No. 5,328,470, 1994), or by stereotactic injection (Chen et al., 1994). The pharmaceutical preparation of a gene therapy vector can include an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

### 9. Dosage

The pharmaceutical composition and method of the present invention may further comprise other therapeutically active compounds as noted herein which are usually applied in the treatment of AAP-related conditions.

In the treatment or prevention of conditions which require AAP modulation an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

However, the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### 10. Kits for Pharmaceutical Compositions

The pharmaceutical compositions can be included in a kit, container, pack, or dispenser together with instructions for administration. When the invention is supplied as a kit, the different components of the composition may be packaged in separate containers and admixed immediately before use. Such packaging of the components separately may permit long-term storage without losing the active components' functions.

Kits may also include reagents in separate containers that facilitate the execution of a specific test, such as diagnostic tests or tissue typing. For example, AAP DNA templates and suitable primers may be supplied for internal controls.

(a) Containers or Vessels

The reagents included in kits can be supplied in containers of any sort such that the life of the different components are preserved, and are not adsorbed or altered by the materials of the container. For example, sealed glass ampules may contain lyophilized AAP or buffer that have been packaged under a neutral, non-reacting gas, such as nitrogen. Ampules may consist of any suitable material, such as glass, organic polymers, such as polycarbonate, polystyrene, etc., ceramic, metal or any other material typically employed to hold reagents. Other examples of suitable containers include simple bottles that may be fabricated from similar substances as ampules, and envelopes, that may consist of foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

(b) Instructional Materials

Kits may also be supplied with instructional materials. Instructions may be printed on paper or other substrate, and/or may be supplied as an electronic-readable medium, such as a floppy disc, CD-ROM, DVD-ROM, Zip disc, videotape, laserdisc, audio tape, etc. Detailed instructions may not be physically associated with the kit; instead, a user may be directed to an internet web site specified by the manufacturer or distributor of the kit, or supplied as electronic mail.

Screening and Detection Methods

The isolated nucleic acid molecules of the invention can be used to express AAP (e.g., via a recombinant expression vector in a host cell in gene therapy applications), to detect AAP mRNA (e.g., in a biological sample) or a genetic lesion in an AAP, and to modulate AAP activity, as described below. In addition, AAP polypeptides can be used to screen drugs or compounds that modulate the AAP activity or expression as well as to treat disorders characterized by insufficient or excessive production of AAP or production of AAP forms that have decreased or aberrant activity compared to AAP wild-type protein, or modulate biological function that involve AAP. In addition, the anti-AAP Abs of the invention can be used to detect and isolate AAP and modulate AAP activity.

### 1. Screening Assays

The invention provides a method (screening assay) for identifying modalities, i.e., candidate or test compounds or agents (e.g., peptides, peptidomimetics, small molecules or other drugs), foods, combinations thereof, etc., that effect AAP, a stimulatory or inhibitory effect, inlcuding translation, transcription, activity or copies of the gene in cells. The invention also includes compounds identified in screening assays.

Testing for compounds that increase or decrease AAP activity are desirable. A compound may modulate AAP activity by affecting: (1) the number of copies of the gene in the cell (amplifiers and deamplifiers); (2) increasing or decreasing transcription of the AAP (transcription up-regulators and down-regulators); (3) by increasing or decreasing the translation of AAP mRNA into protein (translation up-regulators and down-regulators); or (4) by increasing or decreasing the activity of AAP itself (agonists and antagonists).

(a) Effects of Compounds

To identify compounds that affect AAP at the DNA, RNA and protein levels, cells or organisms are contacted with a candidate compound and the corresponding change in AAP DNA, RNA or protein is assessed (Ausubel et al., 1987). For DNA amplifiers and deamplifiers, the amount of AAP DNA is measured, for those compounds that are transcription up-regulators and down-regulators, the amount of AAP mRNA is determined; for translational up- and down-regulators, the amount of AAP polypeptides is measured. Compounds that are agonists or antagonists may be identified by contacting cells or organisms with the compound.

Many assays for screening candidate or test compounds that bind to or modulate the activity of AAP or polypeptide or biologically active portion are available. Test compounds can be obtained using any of the numerous approaches in combinatorial library methods, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptides, while the other four approaches encompass peptide, non-peptide oligomer or small molecule libraries of compounds (Lam, 1997).

(b) Small Molecules

A “small molecule” refers to a composition that has a molecular weight of less than about 5 kD and more preferably less than about 4 kD, and most preferably less than 0.6 kD. Small molecules can be, nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention. Examples of methods for the synthesis of molecular libraries have been described (Carell et al., 1994a; Carell et al., 1994b; Cho et al., 1993; DeWitt et al., 1993; Gallop et al., 1994; Zuckermann et al., 1994).

Libraries of compounds may be presented in solution (Houghten et al., 1992) or on beads (Lam et al., 1991), on chips (Fodor et al., 1993), bacteria, spores (Ladner et al., U.S. Pat. No. 5,223,409, 1993), plasmids (Cull et al., 1992) or on phage (Cwirla et al., 1990; Devlin et al., 1990; Felici et al., 1991; Ladner et al., U.S. Pat. No. 5,223,409, 1993; Scott and Smith, 1990). A cell-free assay comprises contacting AAP or biologically-active fragment with a known compound that binds AAP to form an assay mixture, contacting the assay mixture with a test compound, and determining the ability of the test compound to interact with AAP, where determining the ability of the test compound to interact with AAP comprises determining the ability of the AAP to preferentially bind to or modulate the activity of an AAP target molecule.

(c) Cell-free Assays

The cell-free assays of the invention may be used with both soluble or a membrane-bound forms of AAP. In the case of cell-free assays comprising the membrane-bound form, a solubilizing agent to maintain AAP in solution. Examples of such solubilizing agents include non-ionic detergents such as n-octylglucoside, n-dodecylglucoside, n-dodecylmaltoside, octanoyl-N-methylglucamide, decanoyl-N-methylglucamide, polyoxyethylene ethers (e.g., t-Octylphenoxypolyethoxyethanol (TRITON® X-100) and others from the TRITON® series), polyoxyethylene 9 lauryl ether (THESIT®), Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

(d) Immobilization of Target Molecules to Facilitate Screening

In more than one embodiment of the assay methods, immobilizing either AAP or its partner molecules can facilitate separation of complexed from uncomplexed forms of one or both of the proteins, as well as to accommodate high throughput assays. Binding of a test compound to AAP, or interaction of AAP with a target molecule in the presence and absence of a candidate compound, can be accomplished in any vessel suitable for containing the reactants, such as microtiter plates, test tubes, and micro-centrifuge tubes. A fusion protein can be provided that adds a domain that allows one or both of the proteins to be bound to a matrix. For example, GST (glutathione S-transferase)-AAP fusion proteins or GST-target fusion proteins can be adsorbed onto glutathione sepharose beads (SIGMA Chemical, St. Louis, Mo.) or glutathione derivatized microtiter plates that are then combined with the test compound or the test compound and either the non-adsorbed target protein or AAP, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly. Alternatively, the complexes can be dissociated from the matrix, and the level of AAP binding or activity determined using standard techniques.

Other techniques for immobilizing proteins on matrices can also be used in screening assays. Either AAP or its target molecule can be immobilized using biotin-avidin or biotin-streptavidin systems. Biotinylation can be accomplished using many reagents, such as biotin-NHS (N-hydroxy-succinimide; PIERCE Chemicals, Rockford, Ill.), and immobilized in wells of streptavidin-coated 96 well plates (PIERCE Chemical). Alternatively, Abs reactive with AAP or target molecules but do not interfere with binding of the AAP to its target molecule can be derivatized to the wells of the plate, and unbound target or AAP trapped in the wells by antibody conjugation. Methods for detecting such complexes, in addition to those described for the GST-immobilized complexes, include immunodetection of complexes using Abs reactive with AAP or its target, as well as enzyme-linked assays that rely on detecting an enzymatic activity associated with the AAP or target molecule.

(e) Screens to Identify Modulators

Modulators of AAP expression can be identified in a method where a cell is contacted with a candidate compound and the expression of AAP mRNA or protein in the cell is determined. The expression level of AAP mRNA or protein in the presence of the candidate compound is compared to AAP mRNA or protein levels in the absence of the candidate compound. The candidate compound can then be identified as a modulator of AAP mRNA or protein expression based upon this comparison. For example, when expression of AAP mRNA or protein is greater (i.e., statistically significant) in the presence of the candidate compound than in its absence, the candidate compound is identified as a stimulator of AAP mRNA or protein expression. Alternatively, when expression of AAP mRNA or protein is less (statistically significant) in the presence of the candidate compound than in its absence, the candidate compound is identified as an inhibitor of AAP mRNA or protein expression. The level of AAP mRNA or protein expression in the cells can be determined by methods described for detecting AAP mRNA or protein.

(i) Hybrid Assays

In yet another aspect of the invention, AAP can be used as “bait” in two-hybrid or three hybrid assays (Bartel et al., 1993; Brent et al., WO94/10300, 1994; Iwabuchi et al., 1993; Madura et al., 1993; Saifer et al., U.S. Pat. No. 5,283,317, 1994; Zervos et al., 1993) to identify other proteins that bind or interact with AAP and modulate AAP activity. Such AAP-interacting partner proteins are also likely to be involved in the propagation of signals by the AAP as, for example, upstream or downstream elements of an AAP pathway.

The two-hybrid system is based on the modular nature of most transcription factors, which consists of separable DNA-binding and activation domains. The assay utilizes two different DNA constructs. In one construct, the gene that codes for AAP is fused to a gene encoding the DNA binding domain of a known transcription factor (e.g., GAL4). The other construct, a DNA sequence from a library of DNA sequences that encodes an unidentified protein (“prey” or “sample”) is fused to a gene that codes for the activation domain of the known transcription factor. If the “bait” and the “prey” proteins are able to interact in vivo, forming an AAP-dependent complex, the DNA-binding and activation domains of the transcription factor are brought into close proximity. This proximity allows transcription of a reporter gene (e.g., LacZ) that is operably-linked to a transcriptional regulatory site responsive to the transcription factor. Expression of the reporter gene can be detected, and cell colonies containing the functional transcription factor can be isolated and used to obtain the cloned gene that encodes the AAP-interacting protein.

The invention further pertains to novel agents identified by the aforementioned screening assays and uses thereof for treatments as described herein.

### 2. Detection Assays

Portions or fragments of AAP cDNA sequences (and the complete AAP gene sequences) are useful in themselves. By way of non-limiting example, these sequences can be used to: (1) identify an individual from a minute biological sample (tissue typing); and (2) aid in forensic identification of a biological sample.

(a) Tissue Typing

The AAP sequences of the invention can be used to identify individuals from minute biological samples. In this technique, an individual's genomic DNA is digested with one or more restriction enzymes and probed on a Southern blot to yield unique bands. The sequences of the invention are useful as additional DNA markers for restriction fragment length polymorphisms (RFLP; (Smulson et al., U.S. Pat. No. 5,272,057, 1993)).

Furthermore, the AAP sequences can be used to determine the actual base-by-base DNA sequence of targeted portions of an individual's genome. AAP sequences can be used to prepare two PCR primers from the 5′- and 3′-termini of the sequences that can then be used to amplify an the corresponding sequences from an individual's genome, and the amplified fragment sequenced.

Panels of corresponding DNA sequences from individuals can provide unique individual identifications, as each individual will have a unique set of such DNA sequences due to allelic differences. The sequences of the invention can be used to obtain such identification sequences from individuals and from tissue. The AAP sequences of the invention uniquely represent portions of an individual's genome. Allelic variation occurs to some degree in the coding regions of these sequences, and to a greater degree in the noncoding regions. The allelic variation between individual humans occurs with a frequency of about once ever 500 bases. Much of the allelic variation is due to single nucleotide polymorphisms (SNPs), which include RFLPs.

Each of the sequences described herein can, to some degree, be used as a standard against which DNA from an individual can be compared for identification purposes. Because greater numbers of polymorphisms occur in noncoding regions, fewer sequences are necessary to differentiate individuals. Noncoding sequences can positively identify individuals with a panel of 10 to 1,000 primers that each yield a noncoding amplified sequence of 100 bases. If predicted coding sequences, such as those in SEQ ID NOS:1, 2, 3 or 5 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to treat an individual prophylactically. Accordingly, one aspect of the invention relates to diagnostic assays for determining AAP and/or nucleic acid expression as well as AAP activity, in the context of a biological sample (e.g., blood, serum, cells, tissue) to determine whether an individual is afflicted with a disease or disorder, or is at risk of developing a disorder, associated with aberrant AAP expression or activity, including cancer. The invention also provides for prognostic (or predictive) assays for determining whether an individual is at risk of developing a disorder associated with AAP, nucleic acid expression or activity. For example, mutations in AAP can be assayed in a biological sample. Such assays can be used for prognostic or predictive purpose to prophylactically treat an individual prior to the onset of a disorder characterized by or associated with AAP, nucleic acid expression, or biological activity.

Another aspect of the invention provides methods for determining AAP activity, or nucleic acid expression, in an individual to select appropriate therapeutic or prophylactic agents for that individual (referred to herein as “pharmacogenomics”). Pharmacogenomics allows for the selection of modalities (e.g., drugs, foods) for therapeutic or prophylactic treatment of an individual based on the individual's genotype (e.g., the individual's genotype to determine the individual's ability to respond to a particular agent). Another aspect of the invention pertains to monitoring the influence of modalities (e.g., drugs, foods) on the expression or activity of AAP in clinical trials.

### 1. Diagnostic Assays

An exemplary method for detecting the presence or absence of AAP in a biological sample involves obtaining a biological sample from a subject and contacting the biological sample with a compound or an agent capable of detecting AAP or AAP (e.g., mRNA, genomic DNA) such that the presence of AAP is confirmed in the sample. An agent for detecting AAP mRNA or genomic DNA is a labeled nucleic acid probe that can hybridize to AAP mRNA or genomic DNA. The nucleic acid probe can be, for example, a full-length AAP nucleic acid, such as the nucleic acid of SEQ ID NOS:1, 2, 3 or 5, or a portion thereof, such as an oligonucleotide of at least 15, 30, 50, 100, 250 or 500 nucleotides in length and sufficient to specifically hybridize under stringent conditions to AAP mRNA or genomic DNA.

An agent for detecting AAP polypeptide is an antibody capable of binding to AAP, preferably an antibody with a detectable label. Abs can be polyclonal, or more preferably, monoclonal. An intact antibody, or a fragment (e.g., F_{ab} or F_{(ab′)2}) can be used. A labeled probe or antibody is coupled (i.e., physically linking) to a detectable substance, as well as indirect detection of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently-labeled streptavidin. The term “biological sample” includes tissues, cells and biological fluids isolated from a subject, as well as tissues, cells and fluids present within a subject. The detection method of the invention can be used to detect AAP mRNA, protein, or genomic DNA in a biological sample in vitro as well as in vivo. For example, in vitro techniques for detection of AAP mRNA include Northern hybridizations and in situhybridizations. In vitro techniques for detection of AAP polypeptide include enzyme linked immunosorbent assays (ELISAs), Western blots, immunoprecipitations, and immunofluorescence. In vitro techniques for detection of AAP genomic DNA include Southern hybridizations and fluorescent in situhybridization (FISH). Furthermore, in vivo techniques for detecting AAP include introducing into a subject a labeled anti-AAP antibody. For example, the antibody can be labeled with a radioactive marker whose presence and location in a subject can be detected by standard imaging techniques.

In one embodiment, the biological sample from the subject contains protein molecules, and/or mRNA molecules, and/or genomic DNA molecules. A preferred biological sample is blood.

In another embodiment, the methods further involve obtaining a biological sample from a subject to provide a control, contacting the sample with a compound or agent to detect AAP, AAP mRNA, or genomic DNA, and comparing the presence of AAP, AAP mRNA or genomic DNA in the control sample with the presence of AAP, AAP mRNA or genomic DNA in the test sample.

The invention also encompasses kits for detecting AAP in a biological sample. For example, the kit can comprise: a labeled compound or agent capable of detecting AAP or AAP mRNA in a sample; reagent and/or equipment for determining the amount of AAP in the sample; and reagent and/or equipment for comparing the amount of AAP in the sample with a standard. The compound or agent can be packaged in a suitable container. The kit can further comprise instructions for using the kit to detect AAP or nucleic acid.

### 2. Prognostic Assays

The diagnostic methods described herein can furthermore be utilized to identify subjects having or at risk of developing a disease or disorder associated with aberrant AAP expression or activity. For example, the described assays can be used to identify a subject having or at risk of developing a disorder associated with AAP, nucleic acid expression or activity. Alternatively, the prognostic assays can be used to identify a subject having or at risk for developing a disease or disorder. The invention provides a method for identifying a disease or disorder associated with aberrant AAP expression or activity in which a test sample is obtained from a subject and AAP or nucleic acid (e.g., mRNA, genomic DNA) is detected. A test sample is a biological sample obtained from a subject. For example, a test sample can be a biological fluid (e.g., serum), cell sample, or tissue.

Prognostic assays can be used to determine whether a subject can be administered a modality (e.g., an agonist, antagonist, peptidomimetic, protein, peptide, nucleic acid, small molecule, food, etc.) to treat a disease or disorder associated with aberrant AAP expression or activity. Such methods can be used to determine whether a subject can be effectively treated with an agent for a disorder. The invention provides methods for determining whether a subject can be effectively treated with an agent for a disorder associated with aberrant AAP expression or activity in which a test sample is obtained and AAP or nucleic acid is detected (e.g., where the presence of AAP or nucleic acid is diagnostic for a subject that can be administered the agent to treat a disorder associated with aberrant AAP expression or activity).

The methods of the invention can also be used to detect genetic lesions in an AAP to determine if a subject with the genetic lesion is at risk for a disorder. Methods include detecting, in a sample from the subject, the presence or absence of a genetic lesion characterized by at an alteration affecting the integrity of a gene encoding an AAP polypeptide, or the mis-expression of AAP. Such genetic lesions can be detected by ascertaining: (1) a deletion of one or more nucleotides from AAP; (2) an addition of one or more nucleotides to AAP; (3) a substitution of one or more nucleotides in AAP, (4) a chromosomal rearrangement of an AAP gene; (5) an alteration in the level of an AAP mRNA transcripts, (6) aberrant modification of an AAP, such as a change genomic DNA methylation, (7) the presence of a non-wild-type splicing pattern of an AAP mRNA transcript, (8) a non-wild-type level of AAP, (9) allelic loss of AAP, and/or (10) inappropriate post-translational modification of AAP polypeptide. There are a large number of known assay techniques that can be used to detect lesions in AAP. Any biological sample containing nucleated cells may be used.

In certain embodiments, lesion detection may use a probe/primer in a polymerase chain reaction (PCR) (e.g., (Mullis, U.S. Pat. No. 4,683,202, 1987; Mullis et al., U.S. Pat. No. 4,683,195, 1987), such as anchor PCR or rapid amplification of cDNA ends (RACE) PCR, or, alternatively, in a ligation chain reaction (LCR) (e.g., (Landegren et al., 1988; Nakazawa et al., 1994), the latter is particularly useful for detecting point mutations in AAP-genes (Abravaya et al., 1995). This method may include collecting a sample from a patient, isolating nucleic acids from the sample, contacting the nucleic acids with one or more primers that specifically hybridize to AAP under conditions such that hybridization and amplification of the AAP (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990), transcriptional amplification system (Kwoh et al., 1989); Qβ Replicase (Lizardi et al., 1988), or any other nucleic acid amplification method, followed by the detection of the amplified molecules using techniques well known to those of skill in the art. These detection schemes are especially useful for the detection of nucleic acid molecules present in low abundance.

Mutations in AAP from a sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes can be used to score for the presence of specific mutations by development or loss of a ribozyme cleavage site.

Hybridizing a sample and control nucleic acids, e.g., DNA or RNA, to high-density arrays containing hundreds or thousands of oligonucleotides probes, can identify genetic mutations in AAP (Cronin et al., 1996; Kozal et al., 1996). For example, genetic mutations in AAP can be identified in two-dimensional arrays containing light-generated DNA probes as described (Cronin et al., 1996). Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the AAP and detect mutations by comparing the sequence of the sample AAP-with the corresponding wild-type (control) sequence. Examples of sequencing reactions include those based on classic techniques (Maxam and Gilbert, 1977; Sanger et al., 1977). Any of a variety of automated sequencing procedures can be used when performing diagnostic assays (Naeve et al., 1995) including sequencing by mass spectrometry (Cohen et al., 1996; Griffin and Griffin, 1993; Koster, WO94/16101, 1994).

Other methods for detecting mutations in the AAP include those in which protection from cleavage agents is used to detect mismatched bases in RNA/RNA or RNA/DNA heteroduplexes (Myers et al., 1985). In general, the technique of “mismatch cleavage” starts by providing heteroduplexes formed by hybridizing (labeled) RNA or DNA containing the wild-type AAP sequence with potentially mutant RNA or DNA obtained from a sample. The double-stranded duplexes are treated with an agent that cleaves single-stranded regions of the duplex such as those that arise from base pair mismatches between the control and sample strands. For instance, RNA/DNA duplexes can be treated with RNase and DNA/DNA hybrids treated with S₁ nuclease to enzymatically digest the mismatched regions. In other embodiments, either DNA/DNA or RNA/DNA duplexes can be treated with hydroxylamine or osmium tetroxide and with piperidine in order to digest mismatched regions. The digested material is then separated by size on denaturing polyacrylamide gels to determine the mutation site (Grompe et al., 1989; Saleeba and Cotton, 1993). The control DNA or RNA can be labeled for detection.

Mismatch cleavage reactions may employ one or more proteins that recognize mismatched base pairs in double-stranded DNA (DNA mismatch repair) in defined systems for detecting and mapping point mutations in AAP cDNAs obtained from samples of cells. For example, the mutY enzyme of E. coli cleaves A at G/A mismatches and the thymidine DNA glycosylase from HeLa cells cleaves T at G/T mismatches (Hsu et al., 1994). According to an exemplary embodiment, a probe based on a wild-type AAP sequence is hybridized to a cDNA or other DNA product from a test cell(s). The duplex is treated with a DNA mismatch repair enzyme, and the cleavage products, if any, can be detected from electrophoresis protocols or the like (Modrich et al., U.S. Pat. No. 5,459,039, 1995).

Electrophoretic mobility alterations can be used to identify mutations in AAP. For example, single strand conformation polymorphism (SSCP) may be used to detect differences in electrophoretic mobility between mutant and wild type nucleic acids (Cotton, 1993; Hayashi, 1992; Orita et al., 1989). Single-stranded DNA fragments of sample and control AAP nucleic acids are denatured and then renatured. The secondary structure of single-stranded nucleic acids varies according to sequence; the resulting alteration in electrophoretic mobility allows detection of even a single base change. The DNA fragments may be labeled or detected with labeled probes. The sensitivity of the assay may be enhanced by using RNA (rather than DNA), in which the secondary structure is more sensitive to a sequence changes. The method may use heteroduplex analysis to separate double stranded heteroduplex molecules on the basis of changes in electrophoretic mobility (Keen et al., 1991).

The migration of mutant or wild-type fragments can be assayed using denaturing gradient gel electrophoresis (DGGE; (Myers et al., 1985). In DGGE, DNA is modified to prevent complete denaturation, for example by adding a GC clamp of approximately 40 bp of high-melting GC-rich DNA by PCR. A temperature gradient may also be used in place of a denaturing gradient to identify differences in the mobility of control and sample DNA (Rossiter and Caskey, 1990).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al., 1986; Saiki et al., 1989). Such allele-specific oligonucleotides are hybridized to PCR-amplified target DNA or a number of different mutations when the oligonucleotides are attached to the hybridizing membrane and hybridized with labeled target DNA.

Alternatively, allele specific amplification technology that depends on selective PCR amplification may be used. Oligonucleotide primers for specific amplifications may carry the mutation of interest in the center of the molecule (so that amplification depends on differential hybridization (Gibbs et al., 1989)) or at the extreme 3′-terminus of one primer where, under appropriate conditions, mismatch can prevent, or reduce polymerase extension (Prosser, 1993). Novel restriction site in the region of the mutation may be introduced to create cleavage-based detection (Gasparini et al., 1992). Certain amplification may also be performed using Taq ligase for amplification (Barany, 1991). In such cases, ligation occurs only if there is a perfect match at the 3′-terminus of the 5′ sequence, allowing detection of a known mutation by scoring for amplification.

The described methods may be performed, for example, by using (pre-packaged kits comprising at least one probe (nucleic acid or antibody) that may be conveniently used, for example, in clinical settings to diagnose patients exhibiting symptoms or family history of a disease or illness involving AAP.

Furthermore, any cell type or tissue in which AAP is expressed may be utilized in the prognostic assays described herein.

### 3. Pharmacogenomics

Agents, or modulators that have a stimulatory or inhibitory effect on AAP activity or expression, as identified by a screening assay, can be administered to individuals to treat prophylactically or therapeutically disorders. In conjunction with such treatment, the pharmacogenomics (i.e., the study of the relationship between a subject's genotype and the subject's response to a foreign modality, such as a food, compound or drug) may be considered. Metabolic differences of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Pharmacogenomics can further be used to determine appropriate dosages and therapeutic regimens. Accordingly, the activity of AAP, expression of AAP, or AAP mutation(s) in an individual can be determined to guide the selection of appropriate agent(s) for therapeutic or prophylactic treatment.

Pharmacogenomics deals with clinically significant hereditary variations in the response to modalities due to altered modality disposition and abnormal action in affected persons (Eichelbaum and Evert, 1996; Linder et al., 1997). In general, two pharmacogenetic conditions can be differentiated: (1) genetic conditions transmitted as a single factor altering the interaction of a modality with the body (altered drug action) or (2) genetic conditions transmitted as single factors altering the way the body acts on a modality (altered drug metabolism). These pharmacogenetic conditions can occur either as rare defects or as nucleic acid polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) explains the phenomena of some patients who show exaggerated drug response and/or serious toxicity after taking the standard and safe dose of a drug. These polymorphisms are expressed in two phenotypes in the population, the extensive metabolizer (EM) and poor metabolizer (PM). The prevalence of PM is different among different populations. For example, the CYP2D6 gene is highly polymorphic and several mutations have been identified in PM, which all lead to the absence of functional CYP2D6. Poor metabolizers due to mutant CYP2D6 and CYP2C19 frequently experience exaggerated drug responses and side effects when they receive standard doses. If a metabolite is the active therapeutic moiety, PM shows no therapeutic response, as demonstrated for the analgesic effect of codeine mediated by its CYP2D6-formed metabolite morphine. At the other extreme are the so-called ultra-rapid metabolizers who are unresponsive to standard doses. Recently, the molecular basis of ultra-rapid metabolism has been identified to be due to CYP2D6 gene amplification.

The activity of AAP, expression of AAP nucleic acid, or mutation content of AAP in an individual can be determined to select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an AAP modulator, such as a modulator identified by one of the described exemplary screening assays.

### 4. Monitoring Effects During Clinical Trials

Monitoring the influence of agents (e.g., drugs, compounds) on the expression or activity of AAP can be applied not only in basic drug screening, but also in clinical trials. For example, the effectiveness of an agent determined by a screening assay to increase AAP expression, protein levels, or up-regulate AAP activity can be monitored in clinical trails of subjects exhibiting decreased AAP expression, protein levels, or down-regulated AAP activity. Alternatively, the effectiveness of an agent determined to decrease AAP expression, protein levels, or down-regulate AAP activity, can be monitored in clinical trails of subjects exhibiting increased AAP expression, protein levels, or up-regulated AAP activity. In such clinical trials, the expression or activity of AAP and, preferably, other genes that have been implicated in, for example, cancer can be used as a “read out” or markers for a particular cell's responsiveness.

For example, genes, including AAP, that are modulated in cells by treatment with a modality (e.g., food, compound, drug or small molecule) can be identified. To study the effect of agents on metabolic disorders or disorders associated with changes in adipose tissue physiological function or mass, for example, in a clinical trial, cells can be isolated and RNA prepared and analyzed for the levels of expression of AAP and other genes implicated in the disorder. The gene expression pattern can be quantified by Northern blot analysis, nuclear run-on or RT-PCR experiments, or by measuring the amount of protein, or by measuring the activity level of AAP or other gene products. In this manner, the gene expression pattern itself can serve as a marker, indicative of the cellular physiological response to the agent. Accordingly, this response state may be determined before, and at various points during, treatment of the individual with the agent.

The invention provides a method for monitoring the effectiveness of treatment of a subject with an agent (e.g., an agonist, antagonist, protein, peptide, peptidomimetic, nucleic acid, small molecule, food or other drug candidate identified by the screening assays described herein) comprising the steps of (1) obtaining a pre-administration sample from a subject; (2) detecting the level of expression of an AAP, AAP mRNA, or genomic DNA in the preadministration sample; (3) obtaining one or more post-administration samples from the subject; (4) detecting the level of expression or activity of the AAP, AAP mRNA, or genomic DNA in the post-administration samples; (5) comparing the level of expression or activity of the AAP, AAP mRNA, or genomic DNA in the pre-administration sample with the AAP, AAP mRNA, or genomic DNA in the post administration sample or samples; and (6) altering the administration of the agent to the subject accordingly. For example, increased administration of the agent may be desirable to increase the expression or activity of AAP to higher levels than detected, i.e., to increase the effectiveness of the agent. Alternatively, decreased administration of the agent may be desirable to decrease expression or activity of AAP to lower levels than detected, i.e., to decrease the effectiveness of the agent.

### 5. Methods of Treatment

The invention provides for both prophylactic and therapeutic methods of treating a subject at risk of (or susceptible to) a disorder or having a disorder associated with aberrant AAP expression or activity. Examples include disorders in which cell metabolic demands (and consequently, demands on mitochondria and endoplasmic reticulum) are high, such as during rapid cell growth. Examples of such disorders and diseases include metabolic disorders or disorders associated with changes in adipose tissue physiological function or mass.

### 6. Disease and Disorders

Diseases and disorders that are characterized by increased AAP levels or biological activity, such as obesity, may be treated with therapeutics that antagonize (i.e., reduce or inhibit) activity. Antagonists may be administered in a therapeutic or prophylactic manner. Therapeutics that may be used include: (1) AAP peptides, or analogs, derivatives, fragments or homologs thereof; (2) Abs to AAP; (3) AAP; (4) administration of antisense nucleic acid and nucleic acids that are “dysfunctional” (i.e., due to a heterologous insertion within the coding sequences) that are used to eliminate endogenous function of by homologous recombination (Capecchi, 1989); or (5) modulators (i.e., inhibitors, agonists and antagonists, including additional peptide mimetic of the invention or Abs specific to AAP) that alter the interaction between AAP and its binding partner.

Diseases and disorders that are characterized by decreased AAP levels or biological activity, such as cachexia, may be treated with therapeutics that increase (i.e., are agonists to) activity. Therapeutics that up regulate activity may be administered therapeutically or prophylactically. Therapeutics that may be used include peptides, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying in vitro for RNA or peptide levels, structure and/or activity of the expressed peptides (or AAP mRNAs). Methods include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situhybridization, and the like).

### 7. Prophylactic Methods

The invention provides a method for preventing, in a subject, a disease or condition associated with an aberrant AAP expression or activity, by administering an agent that modulates AAP expression or at least one AAP activity. Subjects at risk for a disease that is caused or contributed to by aberrant AAP expression or activity can be identified by, for example, any or a combination of diagnostic or prognostic assays. Administration of a prophylactic agent can occur prior to the manifestation of symptoms characteristic of the AAP aberrancy, such that a disease or disorder is prevented or, alternatively, delayed in its progression. Depending on the type of AAP aberrancy, for example, an AAP agonist or AAP antagonist can be used to treat the subject. The appropriate agent can be determined based on screening assays.

### 8. Therapeutic Methods

Another aspect of the invention pertains to methods of modulating AAP expression or activity for therapeutic purposes. The modulatory method of the invention involves contacting a cell with an agent that modulates one or more of the activities of AAP activity associated with the cell. An agent that modulates AAP activity can be a nucleic acid or a protein, a naturally occurring cognate ligand of AAP, a peptide, an AAP peptidomimetic, or other small molecule. The agent may stimulate AAP activity. Examples of such stimulatory agents include active AAP and an AAP that has been introduced into the cell. In another embodiment, the agent inhibits AAP activity. Examples of inhibitory agents include antisense AAP nucleic acids and anti-AAP Abs. Modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an AAP or nucleic acid molecule. In one embodiment, the method involves administering an agent (e.g., an agent identified by a screening assay), or combination of agents that modulates (e.g., up-regulates or down-regulates) AAP expression or activity. In another embodiment, the method involves administering an AAP or nucleic acid molecule as therapy to compensate for reduced or aberrant AAP expression or activity.

Stimulation of AAP activity is desirable in situations in which AAP is abnormally down-regulated and/or in which increased AAP activity is likely to have a beneficial effect; for example, in treating cachexia. Conversely, diminished AAP activity is desired in conditions in which AAP activity is abnormally up-regulated and/or in which decreased AAP activity is likely to have a beneficial effect; for example, in treating obesity.

### 9. Determination of the Biological Effect of the Therapeutic

Suitable in vitro or in vivo assays can be performed to determine the effect of a specific therapeutic and whether its administration is indicated for treatment of the affected tissue.

In various specific embodiments, in vitro assays may be performed with representative cells of the type(s) involved in the patient's disorder, to determine if a given therapeutic exerts the desired effect upon the cell type(s). Modalities for use in therapy may be tested in suitable animal model systems including, but not limited to rats, mice, chicken, cows, monkeys, rabbits, dogs and the like, prior to testing in human subjects. Similarly, for in vivo testing, any of the animal model system known in the art may be used prior to administration to human subjects.

### 10. Prophylactic and Therapeutic Uses of the Compositions of the Invention

AAP nucleic acids and proteins are useful in potential prophylactic and therapeutic applications implicated in a variety of disorders including, but not limited to metabolic disorders or disorders associated with changes in adipose tissue physiological function or mass.

As an example, a cDNA encoding AAP may be useful in gene therapy, and the protein may be useful when administered to a subject in need thereof. By way of non-limiting example, the compositions of the invention will have efficacy for treatment of patients suffering from metabolic disorders or disorders associated with changes in adipose tissue physiological function or mass.

AAP nucleic acids, or fragments thereof, may also be useful in diagnostic applications, wherein the presence or amount of the nucleic acid or the protein is to be assessed. A further use could be as an anti-bacterial molecule (i.e., some peptides have been found to possess anti-bacterial properties). These materials are further useful in the generation of Abs that immunospecifically bind to the novel substances of the invention for use in therapeutic or diagnostic methods.

### EXAMPLES

The following examples are included to demonstrate preferred embodiments of the present invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing form the spirit and scope of the invention.

### Example 1

### AAP mRNA Quantitation in Mice and Humans

The abundance of AAP mRNA in mice and human tissues was determined by using quantitative real-time reverse-transcriptase polymerase chain reaction (quantitative RT-PCR) via the TaqMan system (Perkin-Elmer Applied Biosystems, Foster City, Calif.).

Male FVB-N mice, 6 weeks old, were kept at 4° C. for 48 hours to stimulate BAT metabolism, or continually at 22° C. (ambient) or 33° C. to minimize BAT metabolism before sacrificing. BAT, WAT, brain, liver, hindlimb skeletal muscle (skm), lung, kidney, heart, and testis were harvested and mRNA isolated as described below.

Total RNA preparations of individual mice or human subjects were prepared using Ultraspec reagent (Biotecx Laboratories; Houston, Tex.). mRNA abundance was determined following digestion of samples with DNAse as according to the manufacturer's instructions (GIBCO BRL; Grand Island, N.Y.). This system employs primers and probes that are specific to murine or human AAP (Table I). 18S primers/probe, TaqMan reagents, and analytical equipment were purchased from PE Applied Biosystems (Foster City, Calif.). Reactions and detection were carried out using a Model 7700 Sequence Detector in a volume of 50 μL and containing 100 ng RNA, 3 mM MgCl₂, reaction Buffer A (1×), 12.5 U MuLV reverse transcriptase, 1.25 U TaqGold, forward and reverse primers (0.01 O.D. ea.), and 0.1 μM probe (18S analyses employed 240 pg RNA, 5.5 mM MgCl₂, and 0.05 μM probe/primer). Cycling conditions were: 50° C. 15 min and 95° C. 10 min, followed by 40 cycles of 95° C. 15 sec and 60° C. 1 min. 18S mRNA abundance was used as a loading control, and all values reported herein represent 18S-corrected values. Tables II and III summarize the findings.

TABLE I
Primers and probes for AAP

| | SEQ | | | |
|---|---|---|---|---|
| | ID | | | Sequence |
| Species | NO | Type | Designation | (5′->3′) |

| | | | | |
|---|---|---|---|---|
| Human | 7 | Forward | <huy0k0138o1og.forA> | cgccacca |
| | | primer | | cctcctat |
| | | | | gc |
| | 8 | Reverse | <huy0k0138o1og.revA> | aggcgacg |
| | | primer | acggcca | |
| | 9 | Probe | <huy0k0138o1og.probeA> | caagacca |
| | | | agaagccc | |
| | | | ccagagat | |
| | | | tacc | |
| Murine | 10 | Forward | <cgmmy0k0138.forA> | tgttcacc |
| | | primer | | aggatggc |
| | | | | caa |
| | 11 | Reverse | <cgmmy0k0138.revA> | ggttcagc |
| | | primer | | tgccagct |
| | | | | gtatg |
| | 12 | Probe | <cgmmy0k0138.probeA> | ccaaagag |
| | | | | tcactccg |
| | | | | ctcagcca |
| | | | | c |

TABLE II
Distribution and relative abundance of AAP expression in mice

| Tissue | Environmental Condition¹ | mRNA level² |
|---|---|---|
| BAT | 4° C. | 100% |
| | 22° C. | 155% |
| | 33° C. | 172% |
| WAT | 4° C. | 110% |
| | 22° C. | 566% |
| | 33° C. | 434% |
| Brain | 4° C. | <1% |
| | 22° C. | <1% |
| | 33° C. | <1% |
| Liver | 4° C. | <1% |
| | 22° C. | <1% |
| | 33° C. | <1% |
| SKM | 4° C. | <1% |
| | 22° C. | <1% |
| | 33° C. | <1% |
| Lung | 22° C. | <1% |
| Kidney | 22° C. | <1% |
| Heart | 22° C. | <1% |
| Testis | 22° C. | <1% |

¹6 week-old male FVB-N mice were kept at the temperature indicated, except for those kept at 4° C., which was limited to 48 hours.
²Expressed as a percentage of BAT at 4° C.

TABLE III
Distribution and relative abundance of AAP expression in human

| Tissue | mRNA level¹ |
|---|---|
| Skm | <1% |
| Liver | <1% |
| Kidney | <1% |
| Uterus | <1% |
| Testis | <1% |
| Stomach | <1% |
| Spleen | <1% |
| Heart | 6% |
| Lung | <1% |
| Brain | <1% |
| Hibernoma | 29% |
| WAT | 100% |
| Pre-adipocyte | <1% |
| Adipocyte | 207% |

¹Expressed as a percentage of WAT.

When analyzed in mice, AAP mRNA was abundant in murine brown adipose tissue (BAT) and white adipose tissue (WAT), whereas its message was negligible in other tissues, such as lung, heart, kidney, and testis samples in control (22° C.) mice. In human samples, AAP mRNA was especially abundant in the adipose tissue of adult samples, with a specific expression in a BAT-like tumor sample (hibernoma) and in the adipocyte fraction of WAT.

### Example 2

### The AAP Gene is Induced During White Adipose Tissue Adipocyte Development

L1 is a continuous Mus musculus embryonic fibroblast cell line, a substrain of 3T3 (Swiss albino) developed through clonal isolation. 3T3-L1 cells undergo a pre-adipose to adipose like conversion as they progress from a rapidly dividing to a confluent and contact inhibited state. A high serum content in the medium enhances fat accumulation (Green and Meuth, 1974). The cells were induced to convert from pre-adipocytes to adipocytes in differentiation medium, and total mRNA isolated from the cells at days 0, 2, 4, 6 and 8 (n=3/time point) and analyzed for AAP expression. The results are summarized in Table IV.

TABLE IV
AAP expression during differentiation
of fibroblasts to adipocytes in vitro

| Day | Mean¹ |
|---|---|
| 0 | 100% |
| 2 | 176% |
| 4 | 407% |
| 6 | 429% |
| 8 | 390% |

¹Expressed as a percent of the mean (n = 3) day 0 value.

These data indicate that AAP mRNA, and correlatively, protein and activity levels increase during adipogenesis, thus illustrating an important role in adipocyte physiological function.

### Example 3

### Human AAP Variants

At least three variants of human AAP were discovered (SEQ ID NOS:1, 2 and 3) upon sequencing multiple independent clones, each with a different 3′ untranslated region (UTR), and with differences in the length of the 5′ UTR. The open reading frame (ORF) of the variants did not differ, and thus all three transcripts encode the same AAP polypeptide.

### Example 4

### Chromosomal Location of AAP

Radiation hybrid mapping (Bently and Dunham, 1995; Schwartz and Samad, 1997) enabled the determination of chromosomal location of murine and human AAP. The human AAP gene was localized using the human AAP-specific oligo sets (SEQ ID NOS:7, 8 and 9). Real-time RT-PCR was performed on the Stanford G3 Radiation Hybrid Panel (Stewart et al., 1997) of 83 human/hamster somatic clones. Positive clones were identified as having a signal above background and with a threshold below 33 cycles. The data were submitted to the Stanford server (Stewart et al., 1997), and results were returned with linkage on Chromosome 17 with a LOD score of >13. The mouse AAP chromosomal locus was identified through radiation hybrid analysis of 93 hybrids of the Mouse/Hamster T31 Radiation Hybrid Panel (Research Genetics, Huntsville, Ala.), employing PCR primers designed from the mouse AAP fragment sequence (SEQ ID NOS:10 and 11). A collective set of scores (0=no amplification; 1=amplification; 2=uncertain) was used to assess the radiation hybrid vectors, and analyses were performed in duplicate. Chromosomal placement of the mouse AAP gene to Chromosome 11 (LOD>3) was accomplished using the Whitehead Institute/Massachusetts Institute of Technology Center for Genome Research website (Hudson et al., 1995).

Using a probe specific to a portion of the murine AAP 3′ UTR (SEQ ID NO:12), the AAP gene localized near the markers D11Mit322 and D1Mit116 on Chromosome 11 of the mouse, in a region syntenic with human Chromosome 17p13. Consistent with this, mapping efforts using a human radiation hybrid panel identified Chromosome 17p13 as the site of the human AAP gene. Indeed, genetic associations between sites on Chromosome 17 and adipose tissue function or amount have been reported. For instance, person-to-person differences in fat mass (body mass index, or BMI) and levels of plasma leptin (an important adipose-derived hormone whose concentration correlates with WAT mass) are associated with Chromosome 17 in humans (Kissebah et al., 2000; Mitchell et al., 1999).

Equilvalents

Although particular embodiments have been disclosed herein in detail, this has been done by way of example for purposes of illustration only, and is not intended to be limiting with respect to the scope of the appended claims that follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

1. An isolated polypeptide comprising an amino acid sequence having at least 80% sequence identity to the sequence of SEQ ID NOS:4 or 6.

2. The polypeptide of claim 1, wherein said polypeptide is an active AAP polypeptide.

3. The polypeptide of claim 2, wherein said amino acid sequence has at least 90% sequence identity to the sequence of SEQ ID NOS:4 or 6.

4. The polypeptide of claim 2, wherein said amino acid sequence has at least 98% sequence identity to the sequence of SEQ ID NOS:4 or 6.

5. An isolated polynucleotide encoding the polypeptide of any one of claims 1-4, or a complement of said polynucleotide.

6. An isolated polynucleotide comprising a nucleotide sequence having at least 80% sequence identity to the sequence of SEQ ID NOS:1, 2, 3 or 5, or a complement of said polynucleotide.

7. The polynucleotide of claim 6, wherein said nucleotide sequence has at least 90% sequence identity to the sequence of SEQ ID NOS:1, 2, 3 or 5, or a complement of said polynucleotide.

8. The polynucleotide of claim 6, wherein said nucleotide sequence has at least 98% sequence identity to the sequence of SEQ ID NOS:1, 2, 3 or 5, or a complement of said polynucleotide.

9. An antibody that specifically binds to the polypeptide of any one of claims 1-4.

10. A method of treating metabolic disorders comprising modulating the activity of AAP.

11. The method of claim 10 wherein said modulating activity of AAP comprises decreasing the activity of AAP.

12. The method of claim 11, wherein said decreasing activity comprises decreasing the expression of AAP.

13. The method of claim 12, wherein said decreasing expression comprises transforming a cell to express a polynucleotide anti-sense to at least a portion of an endogenous polynucleotide encoding AAP.

14. The method of claim 12, wherein said decreasing activity comprises transforming a cell to express an aptamer to AAP.

15. The method of claim 12, wherein said decreasing activity comprises introducing into a cell an aptamer to AAP.

16. The method of claim 12, wherein said decreasing activity comprises administering to a cell an antibody that selectively binds AAP.

17. The method of claim 12, wherein said decreasing activity comprises disrupting an AAP gene.

18. The method of claim 10 wherein said modulating activity of AAP comprises increasing the activity of AAP.

19. The method of claim 18, wherein said increasing activity comprises increasing the expression of AAP.

20. The method of claim 19, wherein said increasing expression of AAP comprises transforming a cell with an AAP polynucleotide.

22. The method claim 18, wherein said increasing activity comprises administering to a cell an antibody that selectively binds AAP.

23. The method of claim 10, wherein said modulation comprises controlling AAP gene expression with an exogenous promoter.

24. The method of claim 23, wherein said controlling comprises operably-linking the promoter to an endogenous AAP gene.

25. The method of claim 23, wherein said controlling comprises transforming a cell with an AAP gene operably-linked to a promoter.

26. The method of claim 23-25, wherein said promoter is an inducible promoter.

27. A method of treating a metabolic disorder, or a disorder associated with changes in adipose tissue physiological function or mass, comprising:
treating a metabolic disorder or disorder associated with changes in adipose tissue physiological function or massous tumor by any one of the methods of claims 10-26.

28. A method of detecting a metabolic disorder, or a disorder associated with changes in adipose tissue physiological function or mass, comprising:
detecting a change in expression or activity of AAP.

29. The method of claim 27 or 28, wherein said metabolic disordere is associated with an upregulation of AAP activity.

30. The method of claim 29, wherein said metabolic disorder is selected from the group consisting of obesity, tumors, cancers, viral infections and bacterial infections.

31. The method of claim 27 or 28, wherein said metabolic disorder is associated with a down regulation of AAP activity.

32. The method of claim 31, wherein said metabolic disorder is selected from the group consisting of cachexia, tumors, cancers, viral infections and bacterial infections.

33. A method for determining whether a compound up-regulates or down-regulates the transcription of an AAP gene, comprising:
contacting said compound with a composition comprising a RNA polymerase and said gene and measuring the amount of AAP gene transcription.

34. The method of claim 33, wherein said composition is in a cell.

35. A method for determining whether a compound up-regulates or down-regulates the translation of an AAP gene, comprising:
contacting said compound with a composition comprising a ribosome and a polynucleotide corresponding to a mRNA of said gene and measuring the amount of AAP gene translation.

36. The method of claim 35, wherein said composition is in a cell.

37. A vector, comprising a polynucleotide encoding the polypeptide of any one of claims 5-8.

38. A cell, comprising the vector of claim 37.

39. A method of screening a tissue sample for the presence of cells likely to differentiate into adipocytes, comprising:
measuring expression of AAP in said tissue sample.

40. The method of claim 39, wherein said measuring expression of AAP is measuring an amount of AAP.

41. The method of claim 40, wherein said measuring expression of AAP is measuring an amount of mRNA encoding AAP.

42. A transgenic non-human animal, having a disrupted AAP gene.

43. The transgenic non-human animal of claim 42, wherein the non-human animal is a mouse.

44. A transgenic non-human animal, comprising an exogenous polynucleotide having at least 80% sequence identity to the sequence of SEQ ID NOS:1, 2, 3 or 5, or a complement of said polynucleotide.

45. The transgenic non-human animal of claim 44, wherein said exogenous polynucleotide has at least 90% sequence identity to the sequence of SEQ ID NOS:1, 2, 3 or 5, or a complement of said polynucleotide.

46. The transgenic non-human animal of claim 44, wherein said exogenous polynucleotide has at least 98% sequence identity to the sequence of SEQ ID NOS:1, 2, 3 or 5, or a complement of said polynucleotide.

47. A method of screening a sample for an AAP gene mutation, comprising:
comparing an AAP nucleotide sequence in the sample with SEQ ID NOS:4 or 6.

48. A method of determining if a cell is likely to become an adipocyte, comprising:
measuring expression of AAP in said cell.

49. A method of inhibiting adipocyte differentiation, comprising inhibiting the activity of AAP.

50. The method of claim 49, wherein said inhibiting activity of AAP comprises any of the methods of claims 12-17.

51. A method to measuring AAP agonist or antagonist activity of a compound comprising:
contacting the compound with a composition comprising a polypeptide having at least 80% sequence identity to the sequence of SEQ ID NO: 4 or SEQ ID NO:6 and determining if AAP activity is changed.

52. The method of claim 51, wherein said AAP is in a cell.

53. The method of claim 51, wherein said AAP activity is an increase in adipose cell differentiation.

54. The method of claim 51, wherein said AAP activity is a decrease in adipose cell differentiation.


SEQUENCE LISTING (12 sequences)

SEQ ID NO: 1 - DNA, 1519 bp, Homo sapiens Adipose Abundant Protein 1.5 kb form
ccctgactgagcctgagcctccctgggaagaggcgcattcttctctctgctctctgagctttccgttgcttccctgcccatctgtgctctcagcagcaccagcaaagttggcctcaaacttgaacaaagctgcaggctccagcgtctttctgggagctccgcgggggcagcaggcaggccccagcctctgaactcagctggcttgagaagcccagcccaacccttccagcacccagccggccctccgtctcctgagggacgcccctgcccccgacctggaggccctcagtctgggctggggaatggcgcgctgaggtccctccagagccccttgtcccagcctggagctgcagccgcgcaaggcccaggcccccatggcccacccggtgcagtccgagtttccttcagcacaggagccaggctccgccgcatccctggacctgccggagatggagatactcctcaccaaggcagagaacaaggatgacaagaccctgaatctgtccaagaccctctcggggcctctggatctggagcagaacggccagggcctacccttcaaggccatctccgaggggcacctggaggccccactgcctcggtccccctcccgggccagctcaaggagggcgtcctccatcgccaccacctcctatgcccaagaccaagaagcccccagagattacctcatcctggccgtcgtcgcctgcttctgccccgtctggcccctcaacctcatccccctcatcatttccatcatgtctcgaagcagcatgcaacagggcaacgtggacggcgcccggaggctgggccgcctggctcggctgctcagcattaccctcatcatcatgggcatcgtcattatcatggtggccgtgaccgtcaacttcacagttcagaagaaataaacttaagcagggagctgggctagcagaggccggccctggccatcgggagagctctgacctgcacaccgcgggaggccagggtgctgactgtcaagcatcccctggaatgggcacagcacagccagggcgctcttccaggcaggcgaggataacttgagagtttcctagggcaccagggacagagctcagaggcccccgaggtgtgtgtaggaggcggaggcccgcagagcacagagcaggagaagggcttgggccctggaggagaaagccattctggacaccaggggacctggacggagggtccccacagcccgtgccccacgccgcctggaggccagaggggtcagtggccctgctgtcccggctccatcttggttctagccgccacctgcatgaacacagtggcccggcttaacgcactaacccagcctctccctgtgtcccacagggagtagcaagacccaccccacactgccttcaccatctacaccagtgacgccgctgtgtgtcttagcatggaaataaataaacctgaatgtacgtgctcgcaaaaaaaaaaaaaaaaaaaaaaaaaaaaaaaaaaaaaaaaaaaaaaaaaaaaaaaaaaaaaa

SEQ ID NO: 2 - DNA, 1889 bp, Homo sapiens Adipose-Abundant Protein cDNA 1.9 kb
gtgtgcgcacacgcacacacacacacacacacacacacacacagcctccctgactgagcctgagcctccctgggaagaggcgcattcttctctctgctctctgagctttccgttgcttccctgcccatctgtgctctcagcagcaccagcaaagttggcctcaaacttgaacaaagctgcaggctccagcgtctttctgggagctccgcgggggcagcaggcaggccccagcctctgaactcagctggcttgagaagcccagcccaacccttccagcacccagccggccctccgtctcctgagggacgcccctgcccccgacctggaggccctcagtctgggctggggaatggcgcgctgaggtccctccagagccccttgtcccagcctggagctgcagccgcgcaaggcccaggcccccatggcccacccggtgcagtccgagtttccttcagcacaggagccaggctccgccgcatccctggacctgccggagatggagatactcctcaccaaggcagagaacaaggatgacaagaccctgaatctgtccaagaccctctcggggcctctggatctggagcagaacggccagggcctacccttcaaggccatctccgaggggcacctggaggccccactgcctcggtccccctcccgggccagctcaaggagggcgtcctccatcgccaccacctcctatgcccaagaccaagaagcccccagagattacctcatcctggccgtcgtcgcctgcttctgccccgtctggcccctcaacctcatccccctcatcatttccatcatgtctcgaagcagcatgcaacagggcaacgtggacggcgcccggaggctgggccgcctggctcggctgctcagcattaccctcatcatcatgggcatcgtcattatcatggtggccgtgaccgtcaacttcacagttcagaagaaataaacttaagcagggagctgggctagcagaggccggccctggccatcgggagagctctgacctgcacaccgcgggaggccagggtgctgactgtcaagcatcccctgtccccaagttccaaaagcacagactcaaagggaaaccccccagtcacccactgtcagctcccatctgacaagaaagcctggagcgaggaaggacagaaacgaaccgatggttgagggattgtcacgggaggaacatgacacccgaagggactctaggtgccctcggagtgccacacatgcccagaccttctcacacccacacaaataggctctgccgtgcactcctacacacacacacacacacacacacgcttgttcacactcagaacccaggacagccacagccaccgcttaggggaagccactgcagatgcccctggaatgggcacagcacagccagggcgctcttccaggcaggcgaggataacttgagagtttcctagggcaccagggacagagctcagaggcccccgaggtgtgtgtaggaggcggaggcccgcagagcacagagcaggagaagggcttgggccctggaggagaaagccattctggacaccaggggacctggacggagggtccccacagcccgtgccccacgccgcctggaggccagaggggtcagtggccctgctgtcccggctccatcttggttctagccgccacctgcatgaacacagtggcccggcttaacgcactaacccagcctctccctgtgtcccacagggagtagcaagacccaccccacactgccttcaccatctacaccagtgacgccgctgtgtgtcttagcatggaaataaataaacctgaatgtacgtgctcgccaaaaaaaaaaaaaaaaaaaaaaaaaaaaaaaaaaaaaaaaaaaaaaaaaaaaaaaaaaaaaaaaa

SEQ ID NO: 3 - DNA, 2703 bp, Homo sapiens Adipose-Abundant Protein cDNA, 2.7 kb form
ctgctctctgagctttccgttgcttccctgcccatctgtgctctcagcagcaccagcaaagttggcctcaaacttgaacaaagctgcaggctccagcgtctttctgggagctccgcgggggcagcaggcaggccccagcctctgaactcagctggcttgagaagcccagcccaacccttccagcacccagccggccctccgtctcctgagggacgcccctgcccccgacctggaggccctcagtctgggctggggaatggcgcgctgaggtccctccagagccccttgtcccagcctggagctgcagccgcgcaaggcccaggcccccatggcccacccggtgcagtccgagtttccttcagcacaggagccaggctccgccgcatccctggacctgccggagatggagatactcctcaccaaggcagagaacaaggatgacaagaccctgaatctgtccaagaccctctcggggcctctggatctggagcagaacggccagggcctacccttcaaggccatctccgaggggcacctggaggccccactgcctcggtccccctcccgggccagctcaaggagggcgtcctccatcgccaccacctcctatgcccaagaccaagaagcccccagagattacctcatcctggccgtcgtcgcctgcttctgccccgtctggcccctcaacctcatccccctcatcatttccatcatgtctcgaagcagcatgcaacagggcaacgtggacggcgcccggaggctgggccgcctggctcggctgctcagcattaccctcatcatcatgggcatcgtcattatcatggtggccgtgaccgtcaacttcacagttcagaagaaataaacttaagcagggagctgggctagcagaggccggccctggccatcgggagagctctgacctgcacaccgcgggaggccagggtgctgactgtcaagcatcccctgtccccaagttccaaaagcacagactcaaagggaaaccccccagtcacccactgtcagctcccatctgacaagaaagcctggagcgaggaaggaaagcacagcgaacccaatgggtaacgtggtttactctcccggacgcgtcctgggatctcaaccccagcagtctggcttgtttctcattcccacaatttcctgggttccaccaagcagcgaaaactgccaggatgaatgaggaaaaaacccagccccacaaacgagacacacgctggcggggagagacgcagcagagctccttcctgtctgtggactcggagcaaagacgtggggccccatcttttgtgttttcctcaagcggggaaagaatggactgtttgcatgcttcgtgccacacgcccgcggtgatcccagccagggccccgagcgcagaggcggagctgtgctcagcacaggcctgggacctcccccggcaggcacctgtggggggtgcagctcccgggaaggaggcaactgcctcacttaacatcctccgctgcaaggtggtggcgccgagaggcgtgtctgtgaagacaggtaccaggatggcaggacccgcacgcctcttcccacacctgtcagcttcggaagcatctctcgaggactctggtcccaggatgtctcccaggacaagccagtctgcctcttcctcctacttctgctgtagcctgggaccagacctggccaaggtcagccagcggggagggccgaggtctgagctctcgtcctgccgtggcccccgcgatggcctggggtgcaagcttggggctccgtctggtgctgcttaggtagtttcaggcttccagcccagccctatgctctgtgcttggccctgggcccctggccatccacaaacagcagaggccttccctggaggaggctttgggaccacccctggggggacttcccaaagggtcccaggctgtcaggggcgtttgcgccacctgcaagggcgtgggtcagagagtttttatgggggtctgttgtttgaccgtcctgagaccgagggaagggctcagctgaaggaccccgtctgcaggagagtcgggggaccaggtttgggaatacacttgcatgtacagagcctggggcctggacgccacggcctctcatcttaggacctaatgggacccccgaaaggagccaagtggggccctcggcctcttccttcgttccaggcccatgattttccctacacttctccctggcccaggctccagccacaggcacctctcctgcccccgcccaccctcctgaccgcagctcccaggccctggagacctccaggctttcctgccctgggcagccccacctcacagccagagtcaatgccttcatgggaagggctcccagccacacccagagtggcccaaagctgttgaagtcagcatcctttgtcccatcaggaccctcctgcctcctctccaggcccttgttcgcctccccaccctcctcagaggcccggggaagggaagagcaggtcagtacagaggttctgtctacagggaggggccctgggtctatgcacagctggagctctgagccttccacagcccgtgtgactgctagagggcaggggtgcagggctcagtgggcccgggctggtcctttggggctggtgttcctacgtcagtccccacctggggaataaactccagcctctcctgctcatacaaaaaaaaaaaaaaaaaaaaaaaaaaaaaa

SEQ ID NO: 4 - protein, 177 aa, Homo sapiens Adipose-Abundant Protein polypeptide
MAHPVQSEFPSAQEPGSAASLDLPEMEILLTKAENKDDKTLNLSKTLSGPLDLEQNGQGLPFKAISEGHLEAPLPRSPSRASSRRASSIATTSYAQDQEAPRDYLILAVVACFCPVWPLNLIPLIISIMSRSSMQQGNVDGARRLGRLARLLSITLIIMGIVIIMVAVTVNFTVQKK

SEQ ID NO: 5 - DNA, 3102 bp, Mus musculus Adipose-Abundant Protein, cDNA, partial
atgccactccaggtccactgcacctcggttctatcgattcgaattcggccacactggccggatcctctagagatccctcgacctcgacccacgcgtccgggatgaccaagccctgaatctgtccaagtctctgtcaggggctctggacctggagcagaatggtcacagcctgcccttcaaggtcatctccgaggggcaccgccagccctccctttccggctccccttcccgggtcagctctcggcgagcatcctccgtcatcaccacatcctacgcccaggaccaagaagcccccaaagattacctggtccttgccattgcctcttgcttctgccccgtctggcccctcaacctcatcccccttatcttttctatcatgtctcgaagtagtgtgcagcagggggacctggatggggcacggaggctgggccgcctggcacggctactcagcatcaccttcatcatcctgggcatcgttatcatcattgtggctgtcactgtcaatttcacagttccgaagtaaccgcggagctgtttctagcagaggcagaggcagatgccggccagctggagatctccgaacctacatgtccccacacattacaaaggacaggatggtgggtgctgagtgcccacctgtccctaagatccaaaagcacaaacttggagggaaatctctccatcactccattgccagcccacggcagacgggagggaaaatctgaaatgaagttggctgtggactcaatctcagagtcacatctctcgggcttgtttctcatcccacaattcctgggacttacacgtagcgtaaactgccagaatgggaagaaaatcagccccatagccaagaaagaagcactgggtatctatgaacgtgagcacatcctggccgcccgtcttttctatttttattgagggggagtaaagcgactgcttgcatgctttgtgccgagcatccatggtgatcccagccacagcccaaggtgaatctgtcactcagcacaggccctggatacccactggcaaagaggcaacggccatacggaaatcctccattgatgactgactgaatagggatagtatgacagacctggtgctcttgctgaaggtcacgctggaggcttctctaccaaggactctggatccagagtatcttccagaaccacaggatctgcctcttgattttcacctgtgatgtagcctgtggacaccaacctggaagccctagaacttgacctgtcccaggtcagccatgggggagggacagggcttgatctctctctcttgccacggactctttggaggtctgggaccccgtcttggtaacccacacaagtggctcaggagccctatacagggttttcaggggaccctacacagaagagctgaagtagagcctggcttcagactgttagccgagctctaatggatgctctgtgcctggtcctgcatccctggccatccacagcaacaaaggctttccctagaggtagctttgagactattggactctcttgtttattcataggctctggggagctgctgcagcatgtaagggagggggctagagaattcttaggaaggtttgttatttcttgatctggagtccaaggaggggctcacctgaaagggcagtctggggagtggggaccaggacaggaagcaaaggcccagtagccatagcctctcctgagtagaacacggaaaagaagctaatctctgccaaacagcctcatgcttgttgcatctctatttctccacttttttttttttttttttttttttttggcctggcacaggctgtgcccacgggggcttcacctgcttgccttgcctgctctgagctccatctacccaatgtcatactccaccaccaccaacttcagctcagacctggcagaagggcttcccttggccagcagccagcgttttggggggggggggaaacaggtccagggctgtgactacactggtcgggaattggaagttcacttcctctgagcaaaattaggatcctgatccctcctcccttgggccccatgcttggctccctgcgatctcagagaggctgggcagggcacgctagcacagagcactggtcaatgggagagagaaggttggtacccccaaatcaggcccttattgaggaagtgaacttggactcatgttcctaaagcaggggctggctgcacaccctttatgggatctttgaaaccaaactagagatgtcctcgttttctgtgggaagtaagcatgcgagcccagaatacctatgtgacttgagaggttgggtcaggagagatgtgctatctaaagaacagcttatcataaactccagatcctgagtgggtggcctcctaaaggccctcccaggacagatagtgtctagggattgtcatgatggtttctacatcaccagagtccaggtcacccgcagccatttcttctcatctcctagtcacagcctgccagtcacaccttcacccttgcacaccctcacacactcacacattgattcacacttcagcctggaacacgcagggtctctacattagggaatggcttagctagaacattcttctacccaggcaagaacaacctaggagccttcccgtggttccagggacacagcctagaggtccccaagatctcccctgagcagaggggagtacagggcaggaggagagagacattagggtgggaagagagtgagcaagagttctctcagcccccagttcccactgaagctaggagggatgggggccctgccgtcccagctctggcttggttcagctgccagctgtatgagcacagtggctgagcggagtgactctttggcctctctttggccatcctggtgaacagcaagaactgccccctccccaccacacgcactagtgactctgtatttcttggcatggaaagaaataaagctgaatatataaaaaaaaaaaaaaaaaaaaaaaaaaaaaaaaaaaaaaaaaaaaaaaaaaaaaaaaaaaaaaaaagggcggccgcgactctagagtcgacctgcagtagggataacagggtaataagcttggccgccatggcccaacttgtttattgcagcttaaa

SEQ ID NO: 6 - protein, 150 aa, Mus musculus Adipose-Abundant Protein
RSLDLDPRVRDDQALNLSKSLSGALDLEQNGHSLPFKVISEGHRQPSLSGSPSRVSSRRASSVITTSYAQDQEAPKDYLVLAIASCFCPVWPLNLIPLIFSIMSRSSVQQGDLDGARRLGRLARLLSITFIILGIVIIIVAVTVNFTVPK

SEQ ID NO: 7 - DNA, 24 bp, Artificial Sequence, Forward Primer for human AAP
  oligonucleotide primer
gtcattatcatggtggccgtgacc

SEQ ID NO: 8 - DNA, 26 bp, Artificial Sequence; Reverse primer for Human AAP
  oligonucleotide primer
gatgcccatgatgatgagggtaatgc

SEQ ID NO: 9 - DNA, 36 bp, Artificial Sequence, probe for Human AAP
  oligonucleotide probe
ctccagatccagaggccccgagagggtcttggacag

SEQ ID NO: 10 - DNA, 31 bp, Artificial Sequence, Forward primer for Murine AAP
  oligonucleotide primer
ggacttacacgtagcgtaaactgccagaatg

SEQ ID NO: 11 - DNA, 27 bp, Artificial Sequence, Reverse primer for Murine AAP
  oligonucleotide primer
caggaattgtgggatgagaaacaagcc

SEQ ID NO: 12 - DNA, 43 bp, Artificial sequence, Probe for Murine AAP
  oligonucleotide probe
cagccccatagccaagaaagaagcactgggtatctatgaacgt